(19) United States
(12) Patent Application Publication
(10) Pub. No.: US 2026/0125421 A1
(43) Pub. Date: May 7, 2026

(54) ANTIVIRAL COMPOUNDS

(71) Applicant: Hoffmann-La Roche Inc., Little Falls, NJ (US)

(72) Inventors: David James Edmonds, Riehen (CH); Chungen Liang, Shanghai (CN); Hongying Yun, Shanghai (DE); Bo Zhang, Shanghai (CN); Xiufang Zheng, Shanghai (CN)

(73) Assignee: Hoffmann-La Roche Inc., Little Falls, NJ (US)

(21) Appl. No.: 19/120,669

(22) PCT Filed: Oct. 10, 2023
(86) PCT No.: PCT/EP2023/077946

§ 371 Date: Apr. 11, 2025

(30) Foreign Application Priority Data
Oct. 12, 2022 (WO) PCT/CN2022/124828
Nov. 7, 2022 (WO) PCT/CN2022/130336
Apr. 18, 2023 (WO) PCT/CN2023/088962

(51) Int. Cl. C07K 5/103; A61K 38/00; A61P 31/14

(52) U.S. Cl. CPC C07K 5/1008; A61P 31/14; A61K 38/00

(57) ABSTRACT

The invention provides viral protease inhibitors having the general formula (I) wherein the variables are as described herein, compositions including the compounds, processes of manufacturing the compounds and methods of using the compounds.

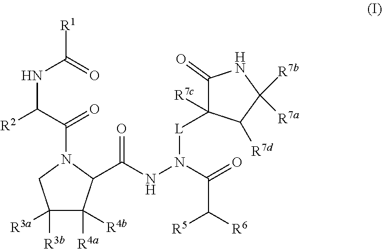

### FIELD OF THE INVENTION

The present invention relates to peptidomimetic compounds that act as viral protease inhibitors. The invention further relates to methods of preparing and using such compounds.

### BACKGROUND OF THE INVENTION

In December 2019, a new coronavirus, named severe acute respiratory syndrome coronavirus 2 (SARS-CoV-2), caused an outbreak of the novel coronavirus disease COVID-19, which has spread to more than 200 countries with over 9 million confirmed cases and over 479,133 confirmed deaths worldwide as of Jun. 26, 2020 (WHO COVID-19 situation report-157). The WHO declared the coronavirus outbreak a public health emergency of international concern. Currently, there are no clinically effective vaccine or specific antiviral drug available for the prevention and treatment of SARS-CoV-2 infections.

Coronaviruses (CoVs) are enveloped, positive-sense, single-stranded RNA viruses. Seven human coronaviruses (HCoVs) have been so far identified, namely HCoV-229E, HCoV-OC43, HCoV-NL63, HCoV-HKU1, SARS coronavirus (SARS-CoV), Middle East respiratory syndrome coronavirus (MERS-CoV) and the novel coronavirus (SARS-CoV-2). While SARS-CoV, MERS-CoV, and SARS-CoV-2 are highly pathogenic, the others generally cause mild to moderate upper-respiratory tract illness and contribute to 15%-30% cases of common colds in human adults.

The RNA genome of SARS-CoV-2 is about 30 kilobases in length shares approximately 80% sequence identity with SARS-CoV (Zhou P. et al. “A pneumonia outbreak associated with a new coronavirus of probable bat origin.” Nature 579(7798): 270-273, 2020). It consists six major open-reading frames (ORFs). ORF 1a/b, which is about two thirds of the whole genome length, directly translates two polyproteins, pp1a and pp1ab, which encodes 16 nonstructural proteins (nsps) to form the replication transcription complex. Nsp3, which encodes papain-like protease (PL^{pro}), and nsp5, which encodes 3-chymotrypsin-like cysteine protease (3CL^{pro}, also known as main protease, M^{pro}), are essential for processing these polyproteins. 3CL^{pro} cleaves the polyprotein at 11 distinct sites to generate various nsps that are important for viral replication. Accordingly, inhibitors that block the cleavage function of 3CL^{pro} could inhibit virus replication. In addition, 3CL^{pro} is highly conserved between SARS-CoV and SARS-CoV-2 (96% sequence identity), as well as the other human coronaviruses. Furthermore, no human proteases with a similar cleavage specificity is known. These desired properties make 3CL^{pro} one of the most attractive targets against coronavirus infections.

### SUMMARY OF THE INVENTION

In a first aspect, the present invention provides compounds of formula (I)

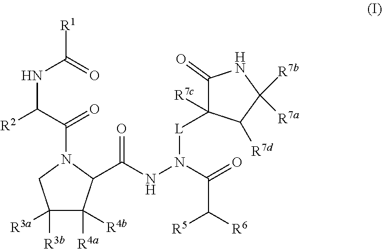

wherein the variables are as defined herein.

In further aspects, the present invention provides processes for manufacturing the compounds of formula (I) described herein, pharmaceutical compositions comprising the same and methods of using the same.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “cycloalkylalkyl” refers to a cycloalkyl group that is bound to the parent molecule via an alkylene group. A particularly preferred, yet non-limiting example of cycloalkylalkyl is 1-bicyclo[1.1.1]pentanylmethyl.

The term “alkylcycloalkyl” refers to a cycloalkyl group, wherein at least one of the hydrogen atoms of the cycloalkyl group has been replaced by an alkyl group. Preferably, “alkylcycloalkyl” refers to a cycloalkyl group wherein 1, 2 or 3 hydrogen atoms of the cycloalkyl group have been replaced by an alkyl group. A particularly preferred, yet non-limiting example of alkylcycloalkyl is 1-methylcyclopropyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “arylalkyl” refers to an aryl group that is bound to the parent molecule via an alkylene group. A particularly preferred, yet non-limiting example of arylalkyl is benzyl.

The term “aryloxy” refers to an aryl group that is bound to the parent molecule via an oxygen atom. A non-limiting example of aryloxy is phenoxy.

The term “heteroaryl” refers to a mono- or multivalent, monocyclic, bicyclic or tricyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered, more preferably a 5-8 membered, yet more preferably a 5-6 membered heteroaryl comprising 1, 2, 3 or 4 heteroatoms independently selected from O, S and N. Most preferably, “heteroaryl” refers to a 5-10 membered, in particular a 5-8 membered or a 5-6 membered heteroaryl comprising 1 to 2 heteroatoms independently selected from O, S and N. In some most preferred instances, “heteroaryl” refers to a 5-6 membered heteroaryl comprising 1 to 2 heteroatoms independently selected from O, S and N. Some non-limiting examples of heteroaryl include spiro[cyclopropane-1,3′-indoline](e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, and morpholinyl. Particularly preferred, yet non-limiting examples of heteroaryl are pyridyl, pyrazinyl, pyrimidinyl, pyrazolyl, imidazolyl, oxazolyl, isoxazolyl, oxadiazolyl and triazolyl.

The term heteroarylalkyl refers to a heteroaryl group that is bound to the parent molecule via an alkylene group. A particularly preferred, yet non-limiting example of heteroarylalkyl is pyridylmethyl.

The term “heterocyclyl” or “heterocycloalkyl” refers to a saturated or partly unsaturated mono- or bicyclic, preferably monocyclic ring system of 3 to 14 ring atoms, preferably 3 to 10 ring atoms, more preferably 3 to 8 ring atoms, most preferably 3 to 6 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, pyrrolidinyl, oxetanyl, 5-azaspiro[2.5]octan-5-yl, piperidyl, 3,3a,4,5,6,6a-hexahydro-1H-cyclopenta[c]pyrrol-2-yl, 2-azaspiro[3.3]heptan-2-yl, 2,6-diazaspiro[3.3]heptanyl, 2-azaspiro[3.5]nonan-2-yl, 1,2-dihydropyridiynl, piperidyl, pyrrolidinyl, and thietanyl.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Some non-limiting examples of haloalkyl include trifluoromethyl, difluoromethyl, CHClF, 1,1-difluoroethyl, 2,2-difluoroethyl, and 2,2,2-trifluoroethyl. Particularly preferred examples of haloalkyl include difluoromethyl and CHClF.

The term “oxo” refers to a double bonded oxygen (═O).

The term “carbamoyl” refers to a group H₂N—C(O)—.

The term “acyl” refers to a group CH₃—C(O)—.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

### Compounds of the Invention

In a first aspect, the present invention provides a compound of Formula (I)

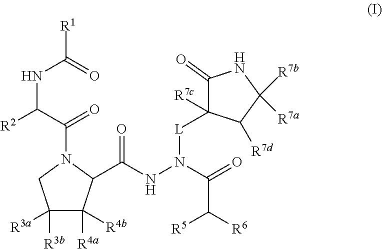

or a pharmaceutically acceptable salt thereof, wherein:
L is C₁-C₆-alkyl;
R¹ is selected from C₃-C₁₀-cycloalkyl, halo-C₃-C₁₀-cycloalkyl, C₆-C₁₄-aryl, 3- to 14-membered heteroaryl, C₁-C₆-alkyl, and halo-C₁-C₆-alkyl;
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from fluoro and chloro;
R⁶ is selected from hydrogen, chloro, and acyl;
R^{7a} and R^{7b} are both C₁-C₆-alkyl, R^{7c} and R^{7d} are both selected from hydrogen and C₁-C₆-alkyl; or
R^{7a}, R^{7b} and R^{7d} are hydrogen and R^{7c} is C₁-C₆-alkyl; or
R^{7a} is C₁-C₆-alkyl, R^{7b} is hydrogen, and R^{7c} and R^{7d} are both selected from hydrogen and C₁-C₆-alkyl; or
R^{7a} and R^{7b}, taken together with the carbon atom to which they are attached, form a C₃-C₁₀-cycloalkyl and R^{7c} and R^{7d} are both selected from hydrogen and C₁-C₆-alkyl; or
R^{7a} and R^{7d}, taken together with the carbon atoms to which they are attached, form a C₃-C₁₀-cycloalkyl and R^{7b} and R^{7c} are both selected from hydrogen and C₁-C₆-alkyl.

In one embodiment, said compound of formula (I) is a compound of formula (Ia)

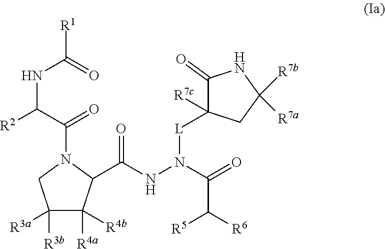

or a pharmaceutically acceptable salt thereof, wherein:
L is C₁-C₆-alkyl;
R¹ is selected from C₃-C₁₀-cycloalkyl, halo-C₃-C₁₀-cycloalkyl, C₆-C₁₄-aryl, 3- to 14-membered heteroaryl, C₁-C₆-alkyl, and halo-C₁-C₆-alkyl;
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from fluoro and chloro;
R⁶ is selected from hydrogen, chloro, and acyl;
R^{7a} and R^{7b} are both C₁-C₆-alkyl and R^{7c} is selected from hydrogen and C₁-C₆-alkyl; or
R^{7a} and R^{7b} are both hydrogen and R^{7c} is C₁-C₆-alkyl; or
R^{7a} is C₁-C₆-alkyl, R^{7b} is hydrogen, and R^{7c} is selected from hydrogen and C₁-C₆-alkyl; or
R^{7a} and R^{7b}, taken together with the carbon atom to which they are attached, form a C₃-C₁₀-cycloalkyl and R^{7c} is selected from hydrogen and C₁-C₆-alkyl.

In one embodiment, the present invention provides a compound of formula (I) as described herein, or a pharmaceutically acceptable salt thereof, wherein R¹ is halo-C₃-C₁₀-cycloalkyl or halo-C₁-C₆-alkyl.

In a preferred embodiment, the present invention provides a compound of formula (I) as described herein, or a pharmaceutically acceptable salt thereof, wherein R¹ is halo-C₃-C₁₀-cycloalkyl.

In one embodiment, the present invention provides a compound of formula (I) as described herein, or a pharmaceutically acceptable salt thereof, wherein R¹ is selected from 1-fluorocyclopropyl, 2,2-difluorocyclopropyl, CHClF, and CHF₂.

In one embodiment, the present invention provides a compound of formula (I) as described herein, or a pharmaceutically acceptable salt thereof, wherein R¹ is selected from 1-fluorocyclopropyl, 2,2-difluorocyclopropyl, and CHF₂.

In a preferred embodiment, the present invention provides a compound of formula (I) as described herein, or a pharmaceutically acceptable salt thereof, wherein R¹ is 1-fluorocyclopropyl or 2,2-difluorocyclopropyl.

In a particularly preferred embodiment, the present invention provides a compound of formula (I) as described herein, or a pharmaceutically acceptable salt thereof, wherein R¹ is 1-fluorocyclopropyl.

In a particularly preferred embodiment, the present invention provides a compound of formula (I) as described herein, or a pharmaceutically acceptable salt thereof, wherein R¹ is 2,2-difluorocyclopropyl.

In one embodiment, the present invention provides a compound of formula (I) as described herein, or a pharmaceutically acceptable salt thereof, wherein R² is C₁-C₆-alkyl.

In a preferred embodiment, the present invention provides a compound of formula (I) as described herein, or a pharmaceutically acceptable salt thereof, wherein R² is selected from tert-butyl, 1-methylpropyl, and 1-ethylpropyl.

In a particularly preferred embodiment, the present invention provides a compound of formula (I) as described herein, or a pharmaceutically acceptable salt thereof, wherein R² is tert-butyl or 1-methylpropyl.

In a particularly preferred embodiment, the present invention provides a compound of formula (I) as described herein, or a pharmaceutically acceptable salt thereof, wherein R² is tert-butyl.

In a particularly preferred embodiment, the present invention provides a compound of formula (I) as described herein, or a pharmaceutically acceptable salt thereof, wherein R² is 1-methylpropyl.

In one embodiment, the present invention provides a compound of formula (I) as described herein, or a pharmaceutically acceptable salt thereof, wherein:
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents.

In a preferred embodiment, the present invention provides a compound of formula (I) as described herein, or a pharmaceutically acceptable salt thereof, wherein:
R^{3a} and R^{4a}, taken together with the carbon atoms to which they are attached, form a cyclopropyl or a cyclopentyl; and R^{3b} and R^{4b} are both hydrogen; wherein said cyclopropyl or cyclopentyl is optionally substituted with 1 to 2 methyl substituents.

In a particularly preferred embodiment, the present invention provides a compound of formula (I) as described herein, or a pharmaceutically acceptable salt thereof, wherein the group

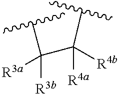

is selected from

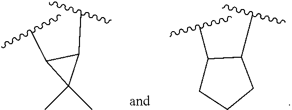

In a particularly preferred embodiment, the present invention provides a compound of formula (I) as described herein, or a pharmaceutically acceptable salt thereof, wherein the group

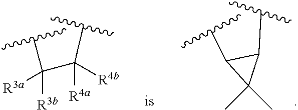

In a particularly preferred embodiment, the present invention provides a compound of formula (I) as described herein, or a pharmaceutically acceptable salt thereof, wherein the group

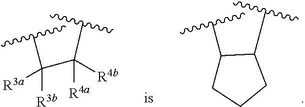

In a preferred embodiment, the present invention provides a compound of formula (I) as described herein, or a pharmaceutically acceptable salt thereof, wherein L is CH₂.

In a preferred embodiment, the present invention provides a compound of formula (I) as described herein, or a pharmaceutically acceptable salt thereof, wherein R⁵ is fluoro and R⁶ is chloro.

In one embodiment, the present invention provides a compound of formula (I) as described herein, or a pharmaceutically acceptable salt thereof, wherein:
R^{7a} and R^{7b} are both C₁-C₆-alkyl and R^{7c} and R^{7d} are both hydrogen; or
R^{7a} and R^{7d}, taken together with the carbon atoms to which they are attached, form a C₃-C₁₀-cycloalkyl and R^{7b} and R^{7c} are both hydrogen; or
R^{7a} is C₁-C₆-alkyl and R^{7b}, R^{7c} and R^{7d} are all hydrogen; or
R^{7a} and R^{7b}, taken together with the carbon atom to which they are attached, form a C₃-C₁₀-cycloalkyl and R^{7c} and R^{7d} are both hydrogen.

In one embodiment, the present invention provides a compound of formula (I) as described herein, or a pharmaceutically acceptable salt thereof, wherein:
R^{7a} and R^{7b} are both methyl and R^{7c} and R^{7d} are both hydrogen; or
R^{7a} and R^{7d}, taken together with the carbon atoms to which they are attached, form a cyclopropyl and R^{7b} and R^{7c} are both hydrogen; or
R^{7a} is methyl and R^{7b}, R^{7c} and R^{7d} are all hydrogen; or
R^{7a} and R^{7b}, taken together with the carbon atom to which they are attached, form a cyclopropyl and R^{7c} and R^{7d} are both hydrogen.

In a preferred embodiment, the present invention provides a compound of formula (I) as described herein, or a pharmaceutically acceptable salt thereof, wherein R^{7a} and R^{7b} are both C₁-C₆-alkyl and R^{7c} is hydrogen.

In a particularly preferred embodiment, the present invention provides a compound of formula (I) as described herein, or a pharmaceutically acceptable salt thereof, wherein R^{7a} and R^{7b} are both methyl and R^{7c} is hydrogen.

In one embodiment, the present invention provides a compound of formula (I) as described herein, or a pharmaceutically acceptable salt thereof, wherein R^{7a} and R^{7b} are both hydrogen and R^{7c} is C₁-C₆-alkyl.

In one embodiment, the present invention provides a compound of formula (I) as described herein, or a pharmaceutically acceptable salt thereof, wherein R^{7a} and R^{7b} are both hydrogen and R^{7c} is methyl.

In one embodiment, the present invention provides a compound of formula (I) as described herein, or a pharmaceutically acceptable salt thereof, wherein R^{7a} is C₁-C₆-alkyl, R^{7b} is hydrogen, and R^{7c} is selected from hydrogen and C₁-C₆-alkyl.

In one embodiment, the present invention provides a compound of formula (I) as described herein, or a pharmaceutically acceptable salt thereof, wherein R^{7a} is C₁-C₆-alkyl and R^{7b} and R^{7c} are both hydrogen.

In one embodiment, the present invention provides a compound of formula (I) as described herein, or a pharmaceutically acceptable salt thereof, wherein R^{7a} is methyl and R^{7b} and R^{7c} are both hydrogen.

In one embodiment, the present invention provides a compound of formula (I) as described herein, or a pharmaceutically acceptable salt thereof, wherein R^{7a} and R^{7b}, taken together with the carbon atom to which they are attached, form a C₃-C₁₀-cycloalkyl and R^{7c} is selected from hydrogen and C₁-C₆-alkyl.

In one embodiment, the present invention provides a compound of formula (I) as described herein, or a pharmaceutically acceptable salt thereof, wherein R^{7a} and R^{7b}, taken together with the carbon atom to which they are attached, form a C₃-C₁₀-cycloalkyl and R^{7c} is hydrogen.

In one embodiment, the present invention provides a compound of formula (I) as described herein, or a pharmaceutically acceptable salt thereof, wherein R^{7a} and R^{7b}, taken together with the carbon atom to which they are attached, form a cyclopropyl and R^{7c} is hydrogen.

In one embodiment, the present invention provides a compound of formula (I) as described herein, or a pharmaceutically acceptable salt thereof, selected from:
N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
rac-(1S)-2,2-difluoro-N-[(1S)-2,2-dimethyl-1-[(3S,3aS,6aR)-3-[[(5,5-dimethyl-2-oxo-pyrrolidin-3-yl)methyl-[(2R)-2-chloro-2-fluoro-acetyl]amino]carbamoyl]-3,3a,4,5,6,6a-hexahydro-1H-cyclopenta[c]pyrrole-2-carbonyl]propyl]cyclopropanecarboxamide;
N-[(1S,2S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
(1S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2R)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2-chloro-2-fluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2-chloro-2-fluoro-acetamide;
(2R)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2R)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2R)-2-chloro-N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2-fluoro-acetamide;
(1S)—N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(2S)-2-chloro-N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2-fluoro-acetamide;
(1S)—N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(6S)-5-oxo-4-azaspiro[2.4]heptan-6-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(6R)-5-oxo-4-azaspiro[2.4]heptan-6-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3S,5S)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R,5S)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R,5R)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S,5R)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3R,5S)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3R,5R)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3S,5S)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3S,5R)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1R,4S,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1S,4S,5S)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(1R,4S,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1R,4R,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(1S,4S,5S)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide; and
(1S)—N-[(1S)-1-[(3S,3aS,6aR)-3-[[[(2S)-2-chloro-2-fluoro-acetyl]-[[(1R,4R,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

In a preferred embodiment, the present invention provides a compound of formula (I) as described herein, or a pharmaceutically acceptable salt thereof, selected from:
N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
rac-(1S)-2,2-difluoro-N-[(1S)-2,2-dimethyl-1-[(3S,3aS,6aR)-3-[[(5,5-dimethyl-2-oxo-pyrrolidin-3-yl)methyl-[(2R)-2-chloro-2-fluoro-acetyl]amino]carbamoyl]-3,3a,4,5,6,6a-hexahydro-1H-cyclopenta[c]pyrrole-2-carbonyl]propyl]cyclopropanecarboxamide;
N-[(1S,2S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide; and
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particularly preferred embodiment, the present invention provides a compound of formula (I) as described herein, or a pharmaceutically acceptable salt thereof, wherein the compound is N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particularly preferred embodiment, the present invention provides a compound of formula (I) as described herein, or a pharmaceutically acceptable salt thereof, wherein the compound is N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particularly preferred embodiment, the present invention provides a compound of formula (I) as described herein, or a pharmaceutically acceptable salt thereof, wherein the compound is (1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

In a particularly preferred embodiment, the present invention provides a compound of formula (I) as described herein, or a pharmaceutically acceptable salt thereof, wherein the compound is (1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

In a particularly preferred embodiment, the present invention provides a compound of formula (I) as described herein, or a pharmaceutically acceptable salt thereof, wherein the compound is rac-(1S)-2,2-difluoro-N-[(1S)-2,2-dimethyl-1-[(3S,3aS,6aR)-3-[[(5,5-dimethyl-2-oxo-pyrrolidin-3-yl)methyl-[(2R)-2-chloro-2-fluoro-acetyl]amino]carbamoyl]-3,3a,4,5,6,6a-hexahydro-1H-cyclopenta[c]pyrrole-2-carbonyl]propyl]cyclopropanecarboxamide.

In a particularly preferred embodiment, the present invention provides a compound of formula (I) as described herein, or a pharmaceutically acceptable salt thereof, wherein the compound is N-[(1S,2S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particularly preferred embodiment, the present invention provides a compound of formula (I) as described herein, or a pharmaceutically acceptable salt thereof, wherein the compound is N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particular embodiment, the present invention provides pharmaceutically acceptable salts of the compounds according to formula (I) as described herein. In a further particular embodiment, the present invention provides compounds according to formula (I) as described herein in their free form (i.e., as free bases or acids).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

All substituents, in particular, R¹ to R⁶, R^{1a}, R^{3a}, R^{3b}, R^{4a}, R^{4b}, R^{7a}, R^{7b}, R^{7c} and L are as defined above and in the claims, unless otherwise indicated.

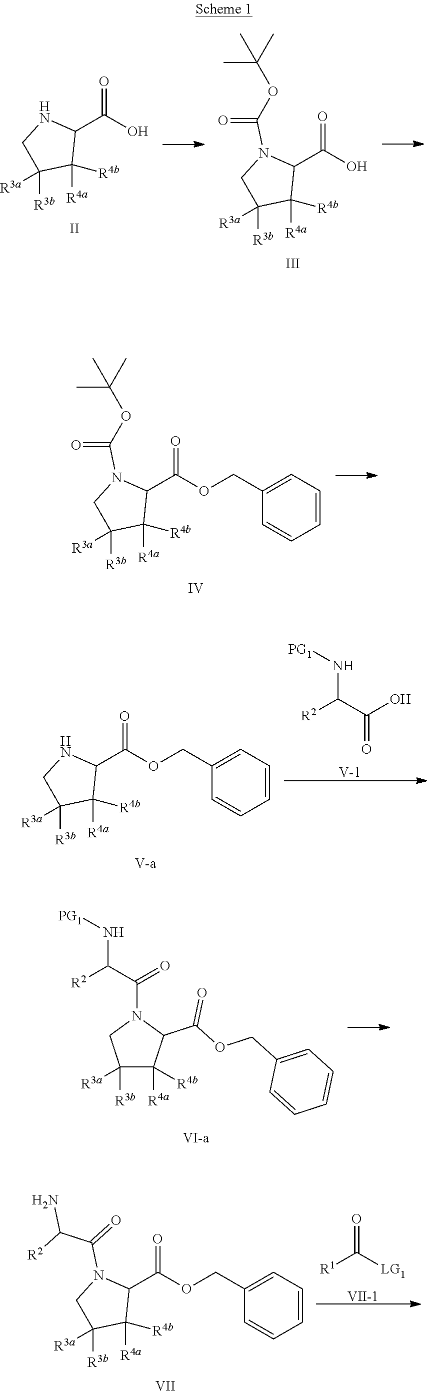

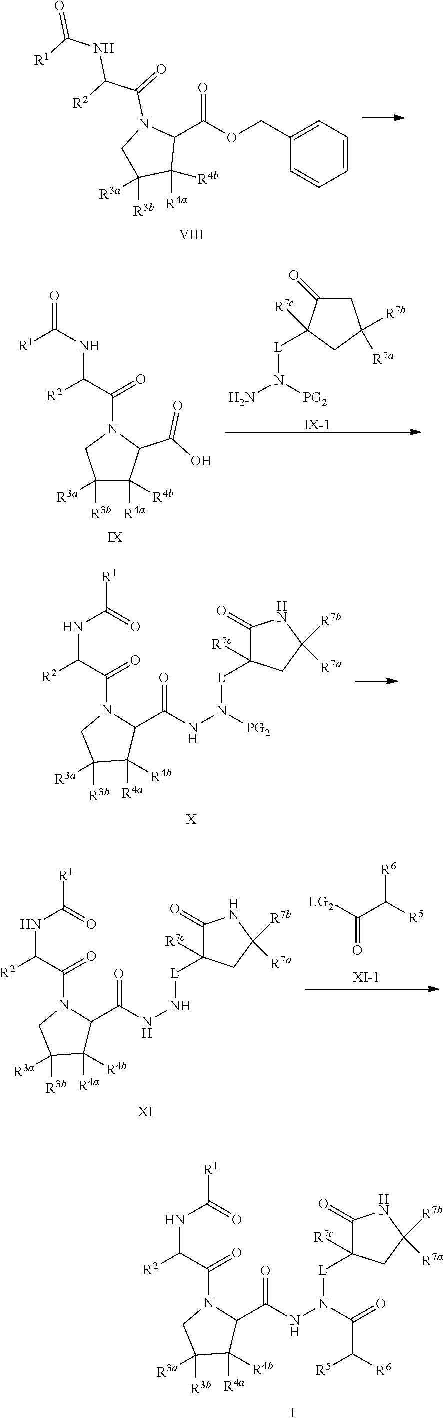

In Scheme 1, PG₁ and PG₂ are protective groups selected from Cbz and Boc, respectively; LG₁ is Cl, OH, OEt or

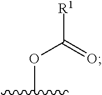

### and LG₂ is Cl or OH.

Compound of formula III can be prepared by a protection reaction of compound of formula II with di-tert-butyl dicarbonate in the presence of an organic base, such as TEA, DIPEA or DMAP, in a solvent such as DCM, THF, dioxane or a mixed solvent of dioxane and water. Then compound of formula III reacts with benzyl bromide in the presence of a base such as Na₂CO₃, K₂CO₃ or Cs₂CO₃, in a solvent such as DMF or CH₃CN, to afford compound of formula IV. Deprotection of compound of formula IV can afford compound of formula V-a in the presence of an acid such as HCl or TFA in a solvent such as DCM or dioxane or a neat reaction without any solvent. Compound of formula VI-a can be obtained by a coupling reaction using compound of formula V-1, compound of formula V-a, and coupling reagent(s), such as T₃P, HATU, HOPO, PyBOP or EDCI/HOBt, in the presence of an organic base, such TEA, DIPEA or DMAP. Deprotection of compound of formula VI-a in the presence of an acid such as HCl or TFA in a solvent such as DCM or dioxane, or a neat reaction without any solvent, can afford compound of formula VII. Compound of formula VIII can be obtained by a coupling reaction using compound of formula VII-1, compound of formula VII, and coupling reagent(s), such as T₃P, HATU, PyBOP, HOPO or EDCI/HOBt, in the presence of an organic base, such TEA, DIPEA or DMAP, in a solvent such as THF, EtOAc, DMF or DCM. Alternatively, compound of formula VIII can be obtained by a reaction of compound of formula VII and compound of formula VII-1 in the presence of an organic base such as TEA, DIPEA or DMAP and in a solvent such as MeOH, DCM, THF or DMF. Hydrogenolysis of compound of formula VIII in the presence of Pd/C, Pd(OH)₂ or a mixture of Pd/C and Pd(OH)₂, and in a solvent such as MeOH, EtOH, THF or EtOAc can afford compound of formula IX. Compound of formula IX reacts with compound of formula IX-1 in the presence of a coupling reagent, such as T₃P, HATU, PyBOP, HOPO or EDCI/HOBt, and an organic base such as TEA, DIPEA or DMAP, in a solvent such as THF, EtOAc, DMF or DCM, to afford compound of formula X. Deprotection of compound of formula X in the presence of an acid such as HCl or TFA in a solvent such as DCM or dioxane, or a neat reaction without any solvent can afford compound of formula XI. Compound of formula XI then reacts with compound of formula XI-1 in the presence (or absence) of coupling reagent(s), such as T₃P, HATU, PyBOP, HOPO or EDCI/HOBt, and a base such as TEA, DIPEA or DMAP, in a solvent such as THF, EtOAc, DMF or DCM, to afford compound of formula I.

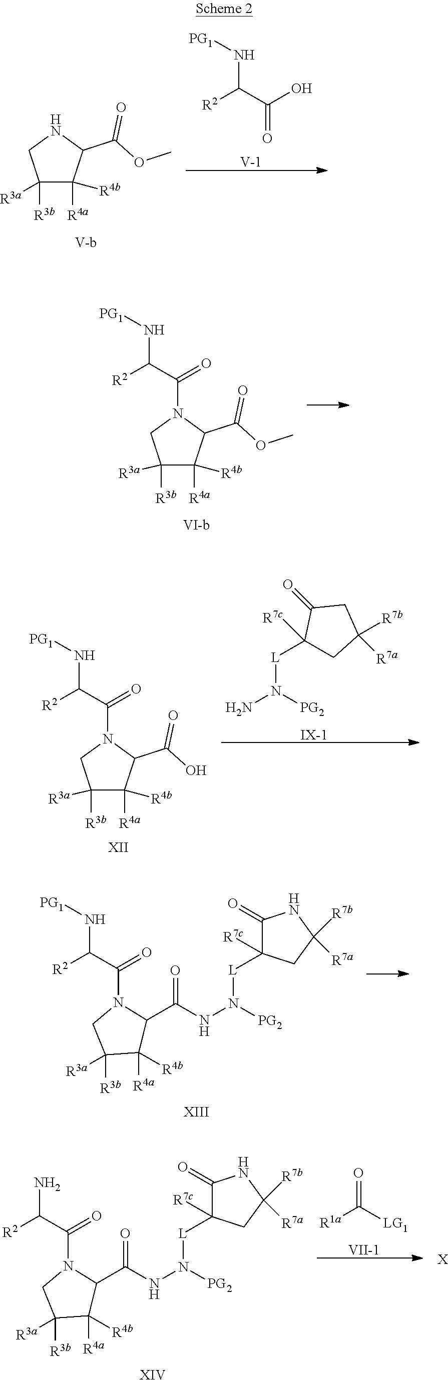

In Scheme 2, PG₁, PG₂ and LG₁ are as defined in Scheme 1.

Alternatively, compound of formula X can be prepared by using compound of formula V-b as starting material. Compound of formula V-b reacts with compound of formula V-1 in the presence of coupling reagent(s), such as T₃P, HATU, PyBOP, HOPO or EDCI/HOBt, and an organic base such as TEA, DIPEA or DMAP, in a solvent such as DMF or DCM, to afford compound of formula VI-b. Hydrolyzation of compound of formula VI-b in the presence of a base such as LiOH H₂O, NaOH or KOH, and in a mixed solution of MeOH and H₂O can afford compound of formula XII. Compound of formula VIII can be obtained by a coupling reaction using compound of formula XII, compound of formula IX-1, and coupling reagent(s), such as T₃P, HATU, HOPO, PyBOP or EDCI/HOBt, in the presence of an organic base, such as TEA, DIPEA or DMAP, in a solvent such as THF, EtOAc, DMF or DCM. Hydrogenolysis of compound of formula XIII in the presence of Pd/C, Pd(OH)₂ or a mixture of Pd/C and Pd(OH)₂ in a solvent such as MeOH, EtOH, THE or EtOAc can afford compound of formula XIV. Compound of formula XIV reacts with compound of formula VII-1 in the presence of coupling reagent(s), such as T₃P, HATU, PyBOP, HOPO or EDCI/HOBt, and an organic base such as TEA, DIPEA or DMAP, in a solvent such as DMF or DCM, to afford compound of formula X. Alternatively, compound of formula X can be obtained by a reaction of compound of formula XIV and compound of formula VII-1 in the presence of an organic base such as TEA, DIPEA or DMAP and in a solvent such as MeOH, DCM, THE or DMF. After deprotection and a coupling reaction, compound of formula I can be prepared from compound of formula X by following the last two steps of Scheme 1.

In one aspect, the present invention provides a process of manufacturing a compound of formula (I) described herein, or a pharmaceutically acceptable salt thereof, comprising: reacting a compound of formula (XI)

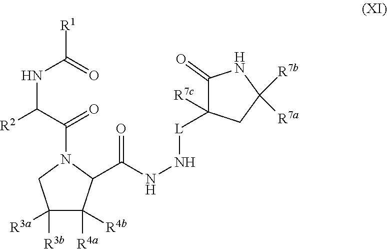

wherein R¹, R², R^{3a}, R^{3b}, R^{4a}, R^{4b}, R^{7a}, R^{7b}, R^{7c}, and L are as defined herein; with a compound of formula (XI-1),

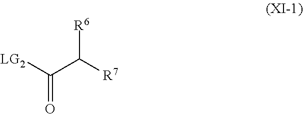

wherein R⁵ and R⁶ are as defined in any one of claims 1 to 16 and LG₂ is a leaving group;
   in the presence of a coupling reagent and a base, to form said compound of formula (I).

In one embodiment, said leaving group LG₂ is a halogen, in particular chloro.

In one embodiment, the base used in said process is selected from TEA, DIPEA and DMAP.

In one embodiment, the solvent used in said process is DMF or DCM.

In one embodiment, the coupling reagent is selected from T₃P, HATU, PyBOP, HOPO and EDCI/HOBt.

### Using the Compounds of the Invention

In one aspect, the present invention provides compounds of formula (I), or pharmaceutically acceptable salts thereof, as described herein for use as therapeutically active substance.

In one aspect, the present invention provides the use of a compound of formula (I) described herein, or a pharmaceutically acceptable salt thereof, for the treatment or prophylaxis of coronavirus infections.

In one aspect, the present invention provides the use of a compound of formula (I) described herein, or a pharmaceutically acceptable salt thereof, for inhibiting the enzymatic activity of 3C-like proteases.

In one aspect, the present invention provides the use of a compound of formula (I) described herein, or a pharmaceutically acceptable salt thereof, for the preparation of a medicament for the treatment or prophylaxis of coronavirus infections.

In one aspect, the present invention provides the use of a compound of formula (I) described herein, or a pharmaceutically acceptable salt thereof, for the preparation of a medicament for inhibiting the enzymatic activity of 3C-like proteases.

In one aspect, the present invention provides a compound of formula (I) described herein, or a pharmaceutically acceptable salt thereof, for use in the treatment or prophylaxis of coronavirus infections.

In one aspect, the present invention provides a compound of formula (I) described herein, or a pharmaceutically acceptable salt thereof, for use in inhibiting the enzymatic activity of 3C-like proteases.

In one aspect, the present invention provides a method of treatment or prophylaxis of coronavirus infections, said method comprising administering a therapeutically active amount of a compound of formula (I) described herein, or a pharmaceutically acceptable salt thereof, to a subject in need.

In one aspect, the present invention provides a method of inhibiting the enzymatic activity of 3C-like proteases, said method comprising contacting a 3C-like protease with a compound of formula (I) described herein, or a pharmaceutically acceptable salt thereof.

In one embodiment, said coronavirus is selected from severe acute respiratory syndrome coronavirus (SARS-CoV), severe acute respiratory syndrome coronavirus 2 (SARS-CoV-2) and Middle East Respiratory Syndrome Coronavirus (MERS-CoV).

In one embodiment, said coronavirus is severe acute respiratory syndrome coronavirus (SARS-CoV).

In one embodiment, said coronavirus is severe acute respiratory syndrome coronavirus 2 (SARS-CoV-2).

In one embodiment, said coronavirus is Middle East Respiratory Syndrome Coronavirus (MERS-CoV).

### Pharmaceutical Compositions and Administration

In one aspect, the present invention provides a pharmaceutical composition comprising a compound of formula (I) as described herein and a therapeutically inert carrier.

In one embodiment, there is provided a pharmaceutical composition according to Example 9 or 10.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Abbreviations

Abbreviations used herein are as follows:
aq. Aqueous
ACN acetonitrile
BnBr benzyl bromide
CbzCl benzyl chloroformate
Cbz benzyl formate
CDCl₃ deuterated chloroform
CD₃OD deuterated methanol
DIPEA N, N-diethylpropylamine
DMF dimethyl formamide
DMSO dimethyl sulfoxide
DBU 1,8-Diazabicycloundec-7-ene
EDCI N-Ethyl-N′-(3-dimethylaminopropyl)carbodiimide hydrochloride
EDTA ethylenediaminetetraacetic acid
EtOAc or EA ethyl acetate
FAM carboxyfluorescein
FRET fluorescence resonance energy transfer
HATU (1-[Bis(dimethylamino)methylene]-1H-1,2,3-triazolo[4,5-b]pyridinium 3-oxid hexafluorophosphate)
HEPES 4-(2-hydroxyethyl)-1-piperazineethanesulfonic acid
h hour
HPLC high performance liquid chromatography
HOBt N-hydroxybenzotriazole
HOPO 2-Hydroxypyridine-N-oxide
LiHMDS lithium bis(trimethylsilyl)amide
N mol/L
MS (ESI) mass spectroscopy (electron spray ionization)
min(s) minute(s)
NMR nuclear magnetic resonance
obsd. Observed
PE Petroleum ether
prep-HPLC preparative high performance liquid chromatography
PyBOP benzotriazol-1-yl-oxytripyrrolidinophosphonium hexafluorophosphate
RT or rt room temperature
sat. saturated
SFC supercritical fluid chromatography
TAMRA carboxytetramethylrhodamine
TCEP tris(2-carboxyethyl)phosphine
TEA triethylamine
TFA trifluoroacetic acid
TFAA trifluoroacetic anhydride
THF tetrahydrofuran
T₃P propylphosphonic anhydride

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Alternatively, intermediates and final compounds were purified by preparative HPLC on reversed phase column using X Bridge™ Perp C₁₈ (5 μm, OBD™ 30×100 mm) column, X Bridge™ Perp C₁₈ (20-40 μm, OBD™ 30×100 mm) column, Welch Ultimate X Bridge™ SiOH 250*50*10 μm column, SunFire™ Perp C₁₈ (5 μm, OBD™ 30×100 mm) column, Phenomenex Luna C18 75*30 mm*3 μm column or Phenomenex Synergi C18 150*25 mm*10 μm column.

For SFC chiral separation, intermediates were separated by chiral column (Daicel chiralpak IC, 5 μm, 30×250 mm), (Daicel chiralpak IC, 10 μm, 30×250 mm), AS (10 μm, 30×250 mm), AD (10 μm, 30×250 mm), Chiralpak IG-3 (50×4.6 mm I.D., 3 μm), using Mettler Toledo Multigram III system SFC, ACSWH-PREP-SFC-C, Waters 80Q preparative SFC or Thar 80 preparative SFC, solvent system: CO₂ and IPA (0.5% TEA in IPA) or CO₂ and MeOH (0.1% NH₃·H₂O in MeOH) or CO₂ and Neu-IPA, back pressure 100 bar, detection UV@254 or 220 nm.

LC/MS spectra were obtained using a Waters UPLC-SQD Mass or SHIMADZU LCMS-2020. Standard LC/MS conditions were as follows (running time 3 mins):
Acidic condition: A: 0.1% formic acid and 1% acetonitrile in H₂O; B: 0.1% formic acid in acetonitrile;
Basic condition: A: 0.05% NH₃·H₂O in H₂O; B: acetonitrile.
Mass spectra (MS): generally only ions which indicate the parent mass are reported, and unless otherwise stated the mass ion quoted is the positive mass ion (M+H)+.
NMR Spectra were obtained by using Bruker Avance 400 MHz.

All reactions involving air-sensitive reagents were performed under an argon atmosphere. Reagents from commercial suppliers were used without further purification unless otherwise noted.

### Preparative Examples

### Intermediate 1

### (2R)-2-chloro-2-fluoro-acetic acid

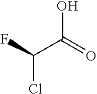

### Step 1: Preparation of 2-chloro-2-fluoroacetic acid

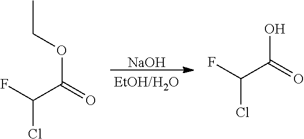

To a solution of ethyl chlorofluoroacetate (1000 g, 7142 mmol) (Aldrich, CAS number: 401-56-9) in ethanol (9 L) was added water (1 L) and NaOH (313 g, 7826 mmol). The reaction mixture was stirred at 25° C. for 12 h. ¹H NMR showed the reaction was complete. The mixture was concentrated in vacuum to remove most of EtOH. Then the residue was diluted with water (1500 mL) and acidified with 2NHCl to pH=4-5. The mixture was extracted with MTBE (1 L×4). The combined organic layers were dried over Na₂SO₄. The mixture was filtered and concentrated to give the crude product 2-chloro-2-fluoroacetic acid (718 g) as colorless oil.

¹H NMR (400 MHz, CDCl₃) δ: ppm 6.16 (d, J=50.4 Hz, 1H).

### Step 2: Preparation of (2R)-2-chloro-2-fluoro-acetic acid

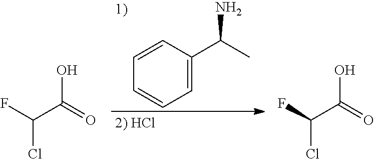

To a solution of 2-chloro-2-fluoro-acetic acid (718.0 g, 3191 mmol) in EtOAc (3000 mL) was added a solution of (S)-1-phenylethanamine (386.7 g, 3191 mmol) in EtOAc (3000 mL) at 0° C. The mixture was stirred at 0° C. for 2 h and then stood overnight. The reaction mixture was filtered and the filter cake was dissolved in acetone (760 g in 7600 mL) at 80° C. The resulting solution was slowly cooled to 20° C. and stood overnight. The precipitate was filtrated, collected and dissolved in acetone (100 g/L) at 80° C. (The recrystallization operation was repeated for 2 times). The collected solid was triturated in acetone for 3 times (acetone, 100 g/500 mL). The solid was collected and dissolved in water (1 L) and acidified with 1M HCl (750 mL). The mixture was extracted with MTBE (1 L×3). The combined organic layers were dried over Na₂SO₄, filtered and concentrated in vacuum to afford (2R)-2-chloro-2-fluoro-acetic acid (121.2 g, 61% purity, Intermediate 1) as yellow oil.

¹H NMR (400 MHz, CDCl₃) δ: ppm 6.29 (d, J=50.8 Hz, 1H).

### Intermediate 2

### (2S)-2-chloro-2-fluoro-acetic acid

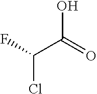

### Preparation of (2S)-2-chloro-2-fluoro-acetic acid

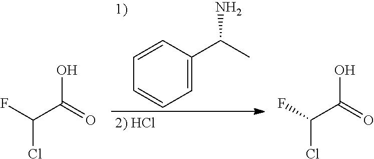

To a solution of 2-chloro-2-fluoro-acetic acid (160.0 g, 1.42 mol) in EtOAc (600 mL) was added (R)-1-phenylethanamine (186.1 g, 1.54 mol) in EtOAc (600 mL) at 0° C. The reaction was stirred at 0° C. for 2 h. The reaction mixture was filtered and the filter cake was triturated in acetone (720 g, 2.2 L) at 80° C. for 1 h. The resulting solution was slowly cooled to 30° C. and stirred at 30° C. for 16 hours. The trituration was repeated for four times. Optical Rotation (C=3.8 g/100 mL in MeOH at 25° C., salt) showed the specific rotation was +10.896. The suspension was filtered, filter cake dissolved in water (1 L), and then acidified with 1NHCl (1.5 L). The mixture was extracted with MTBE (500 mL×10). The combined organic phase was dried over Na₂SO₄, filtered and concentrated to afford (2S)-2-chloro-2-fluoro-acetic acid (138.9 g, Intermediate 2) as a brown liquid.

¹H NMR (400 MHz, CDCl₃) δ: ppm 10.12 (s, 1H), 6.26 (d, J=50.4 Hz, 1H).

### Intermediate 3

### Tert-butyl N-amino-N-[(5,5-dimethyl-2-oxo-pyrrolidin-3-yl)methyl]carbamate

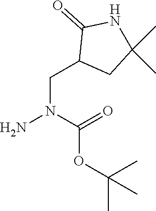 [SMILES: CC1(C)CC(CN(N)C(=O)OC(C)(C)C)C(=O)N1]

### Step 1: Preparation of tert-butyl 3,3-dimethyl-2-oxo-pyrrolidine-1-carboxylate

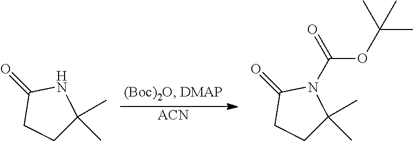

To a solution of 3,3-dimethyl-2-pyrrolidinone (500 mg, 4.42 mmol) in ACN (5 mL) was added DMAP (54 mg, 0.44 mmol) and di-tert-butyldicarbonate (1254 mg, 5.74 mmol). The mixture was stirred at 25° C. for 12 h. The resulting mixture was concentrated in vacuum to give a residue. The residue was purified by silica gel column eluted with PE/EtOAc=4/1 to afford tert-butyl 3,3-dimethyl-2-oxo-pyrrolidine-1-carboxylate (7.1 g) as a white solid.

### Step 2: Preparation of tert-butyl 2,2-dimethyl-5-oxo-pyrrolidine-1-carboxylate

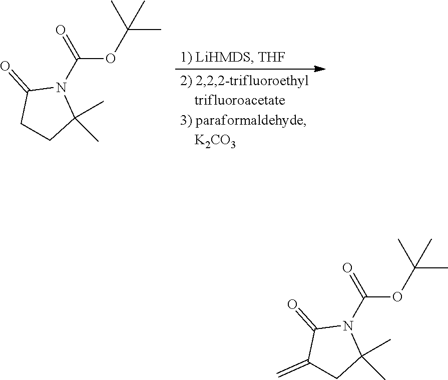 [SMILES: CC(C)(C)OC(=O)N1C(=O)CCC1(C)C ; C=C1CC(C)(C)N(C(=O)OC(C)(C)C)C1=O]

To a solution of tert-butyl 2,2-dimethyl-5-oxo-pyrrolidine-1-carboxylate (7.0 g, 32.82 mmol) in THF (20 mL) was added LiHMDS (36.1 mL, 36.1 mmol) at −78° C. under N₂ atmosphere. The reaction mixture was stirred at the same temperature for 15 min. To the mixture was added 2,2,2-trifluoroethyl trifluoroacetate (6.43 g, 32.82 mmol) at −78° C. Then the mixture was stirred at −78° C. for 15 min. The reaction was quenched with saturated NH₄Cl (20 mL) and extracted with EtOAc (50 mL×3). The organic layers were washed with brine (60 mL), dried over Na₂SO₄, filtered and concentrated in vacuum. The residue was dissolved in toluene (80 mL). To the solution was added paraformaldehyde (4.93 g, 164.11 mmol) and K₂CO₃ (9.98 g, 72.21 mmol). The reaction mixture was stirred at 95° C. for 1 h under N₂ atmosphere. The reaction was diluted with EtOAc (100 mL) and filtered. The filtrate was concentrated in vacuum to afford crude tert-butyl 2,2-dimethyl-5-oxo-pyrrolidine-1-carboxylate (7.0 g) as a yellow solid.

MS obsd. (ESI⁺) [(2M+Na)⁺]: 473.4.

### Step 3: Preparation of tert-butyl 4-[(2-benzyloxycarbonylhydrazino)methyl]-2,2-dimethyl-5-oxo-pyrrolidine-1-carboxylate

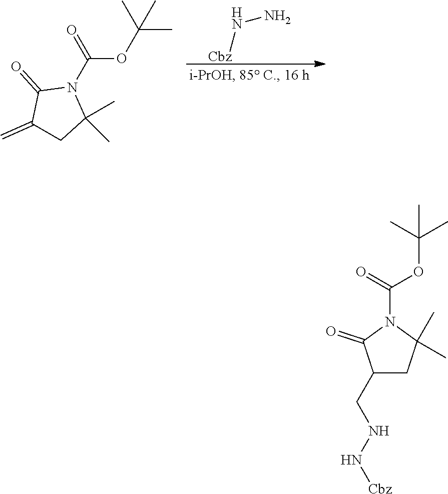 [SMILES: C=C1CC(C)(C)N(C(=O)OC(C)(C)C)C1=O ; NNC(=O)OCc1ccccc1 ; CC(C)(C)OC(=O)N1C(=O)C(CNNC(=O)OCc2ccccc2)CC1(C)C]

To a solution of tert-butyl 2,2-dimethyl-4-methylene-5-oxo-pyrrolidine-1-carboxylate (7.39 g, 32.8 mmol) in IPA (80 mL) was added benzyl carbazate (8.2 g, 49.21 mmol). The mixture was stirred at 85° C. for 12 h. The resulting mixture was concentrated in vacuum to afford tert-butyl 4-[(2-benzyloxycarbonylhydrazino)methyl]-2,2-dimethyl-5-oxo-pyrrolidine-1-carboxylate (12.84 g) as yellow oil.

MS obsd. (ESI⁺) [(2M+H)⁺]: 783.4.

### Step 4: Preparation of benzyl N-[(5,5-dimethyl-2-oxo-pyrrolidin-3-yl)methylamino]carbamate;2,2,2-trifluoroacetic acid

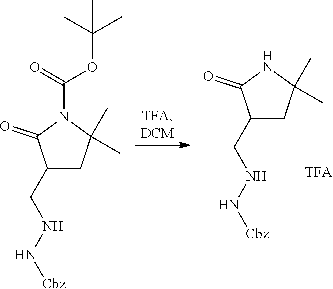 [SMILES: CC(C)(C)OC(=O)N1C(=O)C(CNNC(=O)OCc2ccccc2)CC1(C)C ; CC1(C)CC(CNNC(=O)OCc2ccccc2)C(=O)N1]

A solution of tert-butyl 4-[(2-benzyloxycarbonylhydrazino)methyl]-2,2-dimethyl-5-oxo-pyrrolidine-1-carboxylate (22.2 g, 56.82 mmol) in DCM (80 mL) and TFA (20 mL) was stirred at 25° C. for 1 h. The mixture was concentrated in vacuum to afford benzyl N-[(5,5-dimethyl-2-oxo-pyrrolidin-3-yl)methylamino]carbamate;2,2,2-trifluoroacetic acid (23.0 g) as yellow oil.

MS obsd. (ESI⁺) [(M+H)⁺]: 292.3.

### Step 5: Preparation of tert-butyl N-(benzyloxycarbonylamino)-N-[(2-oxo-3-piperidyl)methyl]carbamate

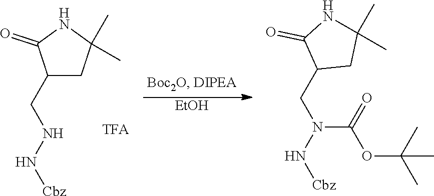 [SMILES: "EtOH" ; CC1(C)CC(CNNC(=O)OCc2ccccc2)C(=O)N1 ; CC1(C)CC(CN(NC(=O)OCc2ccccc2)C(=O)OC(C)(C)C)C(=O)N1]

To a solution of benzyl N-[(5,5-dimethyl-2-oxo-pyrrolidin-3-yl)methylamino]carbamate; 2,2,2-trifluoroacetic acid (23.0 g, 56.74 mmol) in methanol (200 mL) was added DIPEA (73.3 g, 567.38 mmol) and di-tert-butyldicarbonate (37.2 g, 170.21 mmol). The reaction mixture was stirred at 50° C. for 1 h. The mixture was concentrated in vacuum to give a residue. The residue was purified by reversed flash chromatography (condition: 120 g Flash Column Welch Ultimate XB_C18 20-40 μm; 120 A, water (0.10% TFA)-ACN, 0˜60%, 60 ml/min) to afford tert-butyl N-(benzyloxycarbonylamino)-N-[(5,5-dimethyl-2-oxo-pyrrolidin-3-yl)methyl]carbamate (2.5 g) as yellow oil.

MS obsd. (ESI⁺) [(M+H)⁺]: 392.2.

### Step 6: Preparation of tert-butyl N-amino-N-[(2-oxo-3-piperidyl)methyl]carbamate

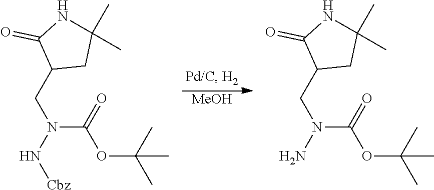 [SMILES: "MeOH" ; CC1(C)CC(CN(NC(=O)OCc2ccccc2)C(=O)OC(C)(C)C)C(=O)N1 ; CC1(C)CC(CN(N)C(=O)OC(C)(C)C)C(=O)N1]

To a solution of tert-butyl N-(benzyloxycarbonylamino)-N-[(5,5-dimethyl-2-oxo-pyrrolidin-3-yl)methyl]carbamate (2.5 g, 6.39 mmol) in methanol (25 mL) was added Pd/C (250 mg, 10% purity). The suspension was degassed under vacuum and purged H₂ for three times. The resulting mixture was stirred at 25° C. under a H₂ balloon for 3 h. The suspension was filtered and the filtrate was concentrated in vacuum to afford tert-butyl N-amino-N-[(5,5-dimethyl-2-oxo-pyrrolidin-3-yl)methyl]carbamate (1.25 g, Intermediate 3) as a yellow solid.

MS obsd. (ESI⁺) [(M+H)⁺]: 258.2.

### Intermediate 4

### Benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate;2,2,2-trifluoroacetic acid

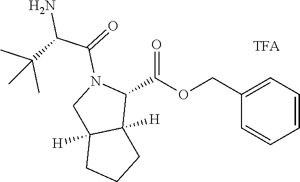 [SMILES: CC(C)(C)[C@H](N)C(=O)N1C[C@@H]2CCC[C@@H]2[C@H]1C(=O)OCc1ccccc1]

### Step 1: Preparation of O3-benzyl O2-tert-butyl (3S,3aS,6aR)-3,3a,4,5,6,6a-hexahydro-1H-cyclopenta[c]pyrrole-2,3-dicarboxylate

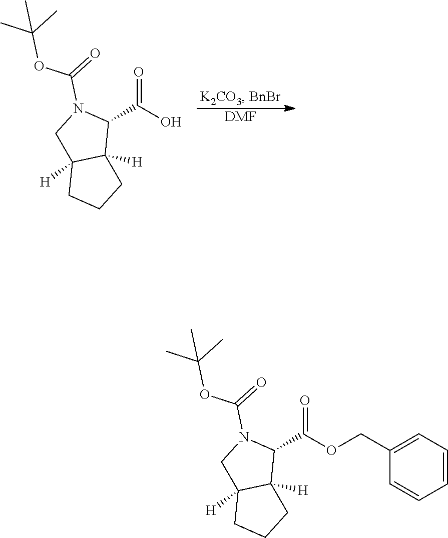

To a solution of (3S,3aS,6aR)-2-tert-butoxycarbonyl-3,3a,4,5,6,6a-hexahydro-1H-cyclopenta[c]pyrrole-3-carboxylic acid (500 mg, 1.96 mmol) in DMF (10 mL) was added K₂CO₃ (541 mg, 3.92 mmol) and BnBr (435 mg, 3.9 mmol). The reaction mixture was stirred at 25° C. for 2 h. The mixture was filtered and the filtrate was concentrated in vacuum. The residue was purified by reverse flash eluted with ACN in H₂O (0.1% TFA)=0˜80% to afford O3-benzyl O2-tert-butyl (3S,3aS,6aR)-3,3a,4,5,6,6a-hexahydro-1H-cyclopenta[c]pyrrole-2,3-dicarboxylate (645 mg) as yellow oil.

MS obsd. (ESI⁺) [(M+Na)⁺]: 368.0.

### Step 2: Preparation of benzyl (3S,3aS,6aR)-1,2,3,3a,4,5,6,6a-octahydrocyclopenta[c]pyrrole-3-carboxylate;trifluoroacetic acid

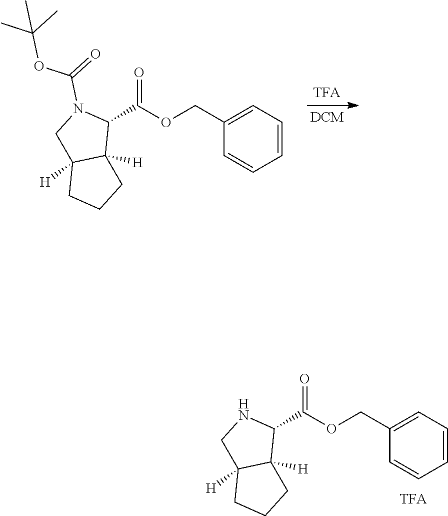 [SMILES: CC(C)(C)OC(=O)N1C[C@@H]2CCC[C@@H]2[C@H]1C(=O)OCc1ccccc1 ; O=C(OCc1ccccc1)[C@H]1NC[C@@H]2CCC[C@@H]21]

A mixture of O3-benzyl O2-tert-butyl (3S,3aS,6aR)-3,3a,4,5,6,6a-hexahydro-1H-cyclopenta[c]pyrrole-2,3-dicarboxylate (645 mg, 1.87 mmol) in DCM (2 mL) and TFA (2 mL) was stirred at 25° C. for 1 h. The mixture was concentrated in vacuum to afford benzyl (3S,3aS,6aR)-1,2,3,3a,4,5,6,6a-octahydrocyclopenta[c]pyrrole-3-carboxylate;trifluoroacetic acid (670 mg, crude) as yellow oil.

MS obsd. (ESI⁺) [(M+H)⁺]: 246.2.

### Step 3: Preparation of (3S,3aS,6aR)-2-[(2S)-2-(tert-butoxycarbonylamino)-3,3-dimethyl-butanoyl]-3,3a,4,5,6,6a-hexahydro-1H-cyclopenta[c]pyrrole-3-carboxylate

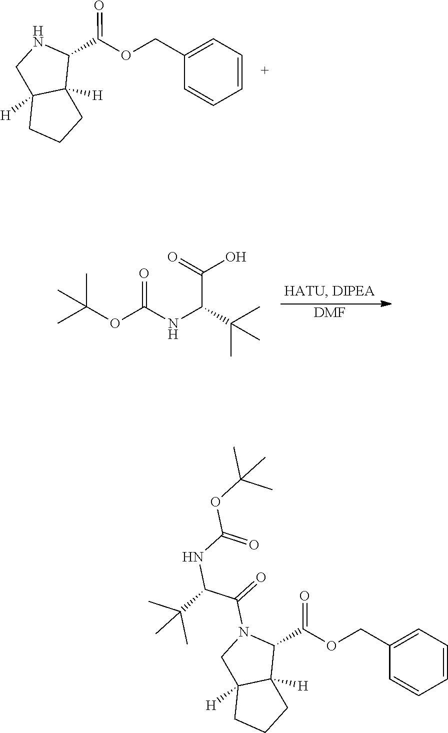

To a mixture of (S)-2-((tert-butoxycarbonyl)amino)-3,3-dimethylbutanoic acid (393 mg, 1.7 mmol) in DMF (5 mL) was added DIPEA (1097 mg, 8.49 mmol), HATU (479 mg, 2.04 mmol) and benzyl (3S,3aS,6aR)-1,2,3,3a,4,5,6,6a-octahydrocyclopenta[c]pyrrole-3-carboxylate;2,2,2-trifluoroacetic acid (610.0 mg, 1.7 mmol) at 0° C. The reaction mixture was stirred at 25° C. for 2 h. The reaction solution was diluted with 40 mL water and extracted with ethyl acetate (50 mL×2). The organic layers were washed with brine (50 mL×2), dried over Na₂SO₄ and concentrated in vacuum. The residue was purified by reverse flash chromatography eluted with ACN in H₂O (0.1% TFA)=0˜86% to afford (3S,3aS,6aR)-2-[(2S)-2-(tert-butoxycarbonylamino)-3,3-dimethyl-butanoyl]-3,3a,4,5,6,6a-hexahydro-1H-cyclopenta[c]pyrrole-3-carboxylate (544 mg) as light yellow oil.

MS obsd. (ESI⁺) [(M+H)⁺]: 459.2.

### Step 4: Preparation of benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate;2,2,2-trifluoroacetic acid

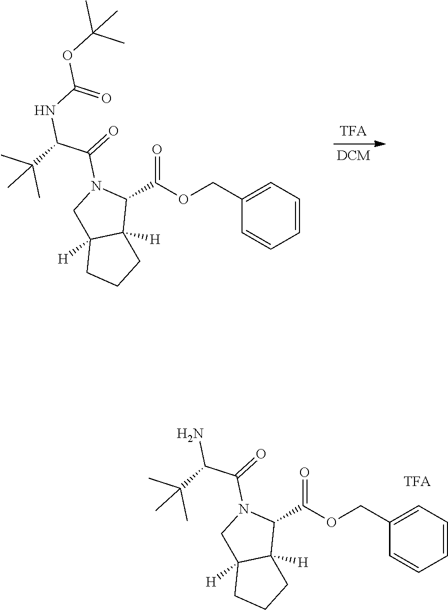 [SMILES: CC(C)(C)OC(=O)N[C@H](C(=O)N1C[C@@H]2CCC[C@@H]2[C@H]1C(=O)OCc1ccccc1)C(C)(C)C ; CC(C)(C)[C@H](N)C(=O)N1C[C@@H]2CCC[C@@H]2[C@H]1C(=O)OCc1ccccc1]

A solution of benzyl (3S,3aS,6aR)-2-[(2S)-2-(tert-butoxycarbonylamino)-3,3-dimethyl-butanoyl]-3,3a,4,5,6,6a-hexahydro-1H-cyclopenta[c]pyrrole-3-carboxylate (544 mg, 1.19 mmol) in DCM (4 mL) and TFA (4 mL) was stirred at 25° C. for 1 h. The resulting mixture was concentrated in vacuum to afford benzyl (3S,3aS,6aR1)-2-[(2S)-2-amino-3,3-dimethyl-butanoyl]-3,3a,4,5,6,6a-hexahydro-1H-cyclopenta[c]pyrrole-3-carboxylate;2,2,2-trifluoroacetic acid (500 mg, Intermediate 4) as yellow oil.

MS obsd. (ESI⁺) [(M+H)⁺]: 359.3.

### Example 1a and 1b

### N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide and N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

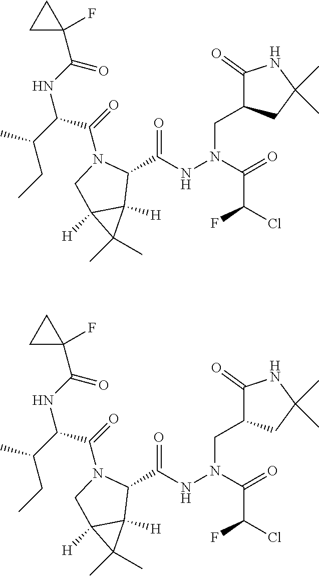

### Step 1: Preparation of methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate

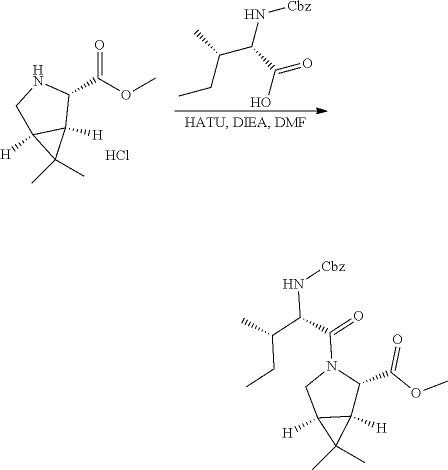

To a solution of Z-ILe-OH (2.84 g, 10.7 mmol, TCI, CAS: 3160-59-6) in DMF (30 mL) was added DIPEA (5.03 g, 38.9 mmol), HATU (4.44 g, 11.67 mmol) and methyl (1R,2S,5S)-6,6-dimethyl-3-azabicyclo[3.1.0]hexane-2-carboxylate;hydrochloride (2.0 g, 9.72 mmol, WuXi AppTec, CAS: 565456-77-1) at 0° C. The reaction mixture was stirred at 25° C. for 2 h. The resulting mixture was diluted with water (50 mL) and extracted with EtOAc (150 mL×3). The combined organic layers were washed with brine (100 mL) and dried over Na₂SO₄, filtered and concentrated. The residue was purified by silica gel column eluted with PE to PE/EtOAc=2/1 to afford methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate (3.03 g) as a yellow solid.

MS obsd. (ESI⁺) [(M+H)⁺]: 417.3.

### Step 2: Preparation of (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid

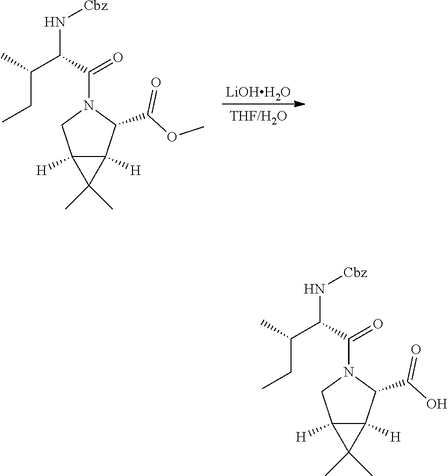

To a solution of methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate (3.0 g, 7.2 mmol) in THE (20 mL) was added a solution of LiOH·H₂O (605 mg, 14.42 mmol) in water (20 mL) at 0° C. The reaction mixture was stirred at 25° C. for 150 min. The resulting mixture was diluted with 1 NHCl (50 mL) and extracted with EtOAc (50 mL×3). The combined organic layers were washed with brine (50 mL), dried over Na₂SO₄, filtered and concentrated in vacuum to afford (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid (2.42 g) as colorless oil.

MS obsd. (ESI⁺) [(M+H)⁺]: 403.2.

### Step 3: Preparation of tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate

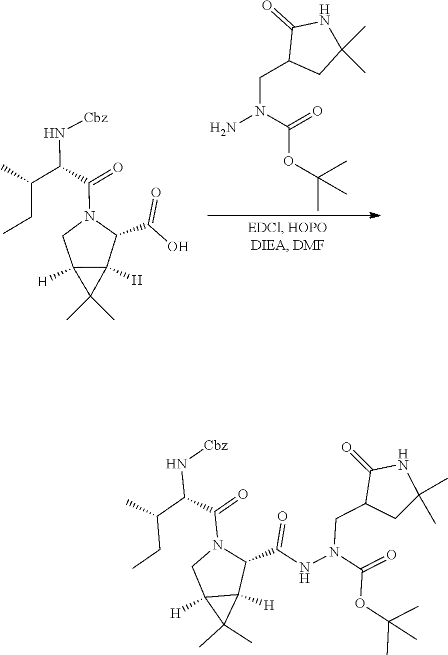 [SMILES: CC[C@H](C)[C@H](NC(=O)OCc1ccccc1)C(=O)N1C[C@H]2[C@@H]([C@H]1C(=O)O)C2(C)C ; CC1(C)CC(CN(N)C(=O)OC(C)(C)C)C(=O)N1 ; CC[C@H](C)[C@H](NC(=O)OCc1ccccc1)C(=O)N1C[C@H]2[C@@H]([C@H]1C(=O)NN(CC1CC(C)(C)NC1=O)C(=O)OC(C)(C)C)C2(C)C]

To a solution of (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid (800 mg, 1.99 mmol) in DMF (10 mL) was added DIPEA (771 mg, 5.96 mmol), HOPO (287 mg, 2.58 mmol), EDCI (495 mg, 2.58 mmol) and tert-butyl N-amino-N-[(5,5-dimethyl-2-oxo-pyrrolidin-3-yl)methyl]carbamate (511 mg, 1.99 mmol, Intermediate 3) at 0° C. Then the mixture was stirred at 25° C. for 12 h. The resulting mixture was diluted with EtOAc (200 mL), washed with 1 NHCl (60 mL) and brine (60 mL), dried over Na₂SO₄ and concentrated in vacuum to afford tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate (1210 mg) as a yellow solid.

MS obsd. (ESI⁺) [(M+H)⁺]: 642.3.

### Step 4: Preparation of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate

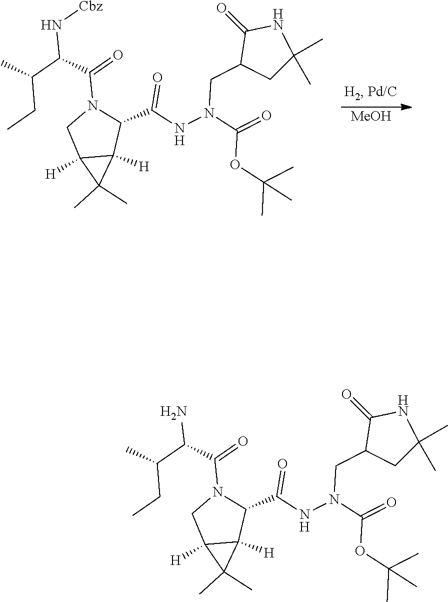

To a solution of tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate (1.2 g, 1.87 mmol) in methanol (30 mL) was added 100 mg of 10% Pd on activated charcoal. The suspension was degassed under vacuum and purged hydrogen for three times. The resulting mixture was stirred at 25° C. under a hydrogen balloon for 2 h. The suspension was filtered and the filtrate was concentrated in vacuum to afford tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate (953 mg) as yellow oil.

MS obsd. (ESI⁺) [(M+H)⁺]: 508.4.

### Step 5: Preparation of tert-butyl N-[rac-(5,5-dimethyl-2-oxo-pyrrolidin-3-yl)methyl]-N-[[(1R,2S,5S)-3-[(2S,3S)-2-[(1-fluorocyclopropanecarbonyl)amino]-3-methyl-pentanoyl]-6,6-dimethyl-3-azabicyclo[3.1.0]hexane-2-carbonyl]amino]carbamate

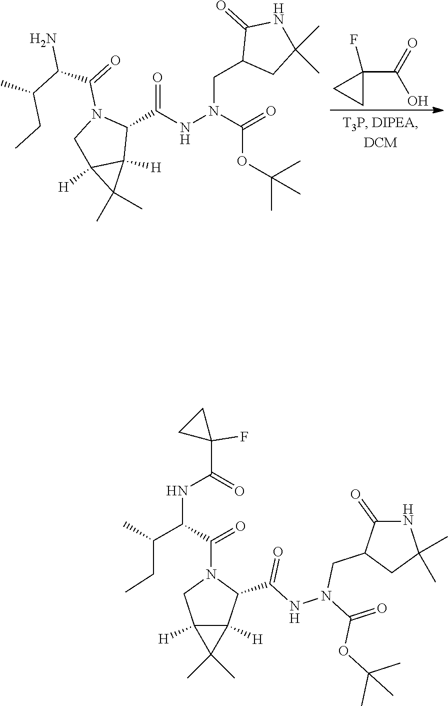

To a solution of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[rac-(5,5-dimethyl-2-oxo-pyrrolidin-3-yl)methyl]carbamate (950 mg, 1.87 mmol) and 1-fluorocyclopropane-1-carboxylic acid (292 mg, 2.8 mmol) in DCM (16 mL) was added DIPEA (726 mg, 5.61 mmol). After cooling to 0° C., to the mixture was added T₃P (1905 mg, 2.99 mmol, 50% in EtOAc). The reaction mixture was stirred at 0° C. for 1 h. The resulting mixture was diluted with DCM (150 mL), washed with brine (60 mL×2), dried over Na₂SO₄, filtered and concentrated in vacuum to afford tert-butyl N-[rac-(5,5-dimethyl-2-oxo-pyrrolidin-3-yl)methyl]-N-[[(1R,2S,5S)-3-[(2S,3S)-2-[(1-fluorocyclopropanecarbonyl)amino]-3-methyl-pentanoyl]-6,6-dimethyl-3-azabicyclo[3.1.0]hexane-2-carbonyl]amino]carbamate (820 mg) as yellow oil.

MS obsd. (ESI⁺) [(M+H)⁺]: 594.3.

### Step 6: Preparation of N-[(1S,2S)-1-[(1R,2S,5S)-2-[[rac-(5,5-dimethyl-2-oxo-pyrrolidin-3-yl)methylamino]carbamoyl]-6,6-dimethyl-3-azabicyclo[3.1.0]hexane-3-carbonyl]-2-methyl-butyl]-1-fluoro-cyclopropanecarboxamide;hydrochloride

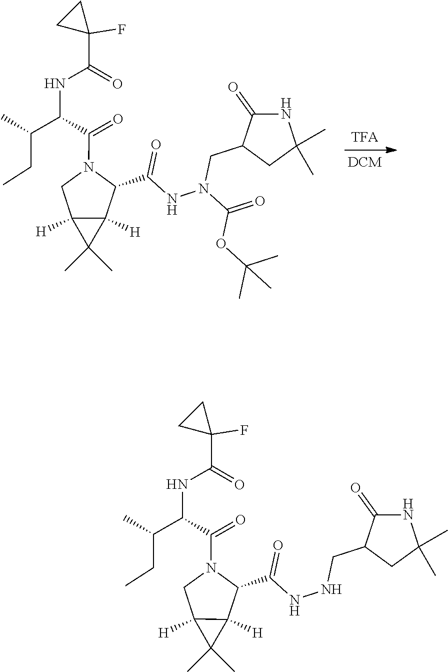

A solution of rac-tert-butyl N-[(5,5-dimethyl-2-oxo-pyrrolidin-3-yl)methyl]-N-[[(1R,2S,5S)-3-[(2S,3S)-2-[(1-fluorocyclopropanecarbonyl)amino]-3-methyl-pentanoyl]-6,6-dimethyl-3-azabicyclo[3.1.0]hexane-2-carbonyl]amino]carbamate (800.0 mg, 1.35 mmol) in DCM (10 mL) and TFA (3 mL) was stirred at 25° C. for 1 h. The resulting mixture was concentrated in vacuum and the residue was purified by reverse flash chromatography (condition: 40 g Flash Column Welch Ultimate XB_C18 20-40 μm; 120 A, water (0.1% HCl)-ACN, 45%, 70 mL/min) to afford N-[(1S,2S)-1-[(1R,2S,5S)-2-[[rac-(5,5-dimethyl-2-oxo-pyrrolidin-3-yl)methylamino]carbamoyl]-6,6-dimethyl-3-azabicyclo[3.1.0]hexane-3-carbonyl]-2-methyl-butyl]-1-fluoro-cyclopropanecarboxamide;hydrochloride (700 mg) as a light yellow solid.

MS obsd. (ESI⁺) [(M+H)⁺]: 494.4.

### Step 7: Preparation of N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide and N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

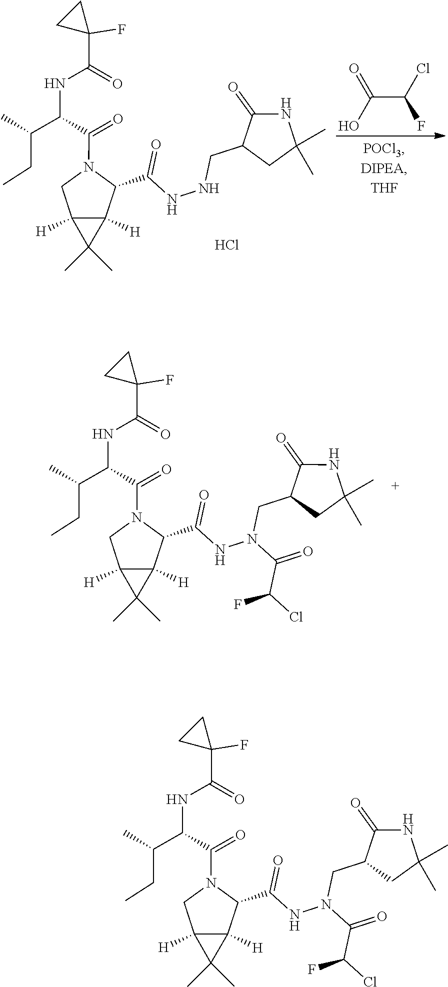

To a solution of (2R)-2-chloro-2-fluoro-acetic acid (424 mg, 2.64 mmol, Intermediate 1) in DCM (10 mL) was added POCl₃ (369 mg, 2.38 mmol). The mixture was stirred at 25° C. for 1 h. Then the resulting solution was added dropwise to a solution of rac-N-[(1S,2S)-1-[(1R,2S,5S)-2-[[(5,5-dimethyl-2-oxo-pyrrolidin-3-yl)methylamino]carbamoyl]-6,6-dimethyl-3-azabicyclo[3.1.0]hexane-3-carbonyl]-2-methyl-butyl]-1-fluoro-cyclopropanecarboxamide;hydrochloride (700 mg, 1.32 mmol) and DIPEA (1365 mg, 10.56 mmol) in DCM (10 mL) at −5° C. Then the mixture was stirred at the same temperature for 30 min. The reaction was quenched with MeOH (10 mL) and acidified by 4 NHCl/dioxane to pH=5 at −10° C. Then the resulting mixture was concentrated in vacuum at 30° C. and the residue was purified by reversed flash chromatography (condition: 40 g Flash Column Welch Ultimate XB_C18 20-40 μm; 120 A, water (0.1% FA)-ACN, 51%, 70 mL/min) to afford a mixture of N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide and N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide. The mixture was split by prep-SFC (Sample preparation: Add CH₃OH 20 mL into sample; Instrument: Waters 80Q; Mobile Phase: 15% MeOH (0.1% NH₃·H₂O) in Supercritical CO₂; Flow Rate: 50 g/min; Cycle Time: 5.8 min, total time: 50 min; Single injection volume: 2.5 mL; Back Pressure: 100 bar to keep the CO₂ in Supercritical flow) to afford Example 1a (139 mg, retention time=6.06 min) and Example 1b (79 mg, retention time=8.83 min) as white solids.

### Example 1a

¹H NMR (400 MHz, DMSO-d₆) δ: ppm 11.09 (s, 1H), 8.55-8.34 (d, J=7.2 Hz, 1H), 7.90 (s, 1H), 6.85 (d, J=50.4 Hz, 1H), 4.26-4.19 (m, 1H), 4.17 (s, 1H), 3.98-3.92 (m, 1H), 3.89-3.79 (m, 2H), 3.52-3.42 (m, 2H), 2.75-2.67 (m, 1H), 2.12-2.03 (m, 1H), 2.01-1.90 (m, 1H), 1.69-1.56 (m, 2H), 1.55-1.44 (m, 2H), 1.34-1.25 (m, 2H), 1.22 (s, 3H), 1.14 (s, 3H), 1.10-1.05 (m, 2H), 1.04 (s, 3H), 0.90 (s, 3H), 0.82 (t, J 7.6 Hz, 6H).

MS obsd. (ESI⁺) [(M+H)⁺]: 588.2.

### Example 1b

¹H NMR (400 MHz, DMSO-d₆) δ: ppm 11.09 (s, 1H), 8.48 (d, J=7.6 Hz, 1H), 7.86 (s, 1H), 6.80 (d, J=50.4 Hz, 1H), 4.21-4.13 (m, 1H), 4.08 (s, 1H), 3.93-3.86 (m, 1H), 3.84-3.74 (m, 2H), 3.39-3.33 (m, 2H), 2.81-2.73 (m, 1H), 2.05-1.85 (m, 2H), 1.60-1.50 (m, 2H), 1.48-1.39 (m, 2H), 1.29-1.18 (m, 2H), 1.15 (s, 3H), 1.11 (s, 3H), 1.06-1.00 (m, 2H), 0.98 (s, 3H), 0.84 (s, 3H), 0.80-0.72 (m, 6H).

MS obsd. (ESI⁺) [(M+H)⁺]: 588.2.

### Example 2a and 2b

### (1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide and (1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

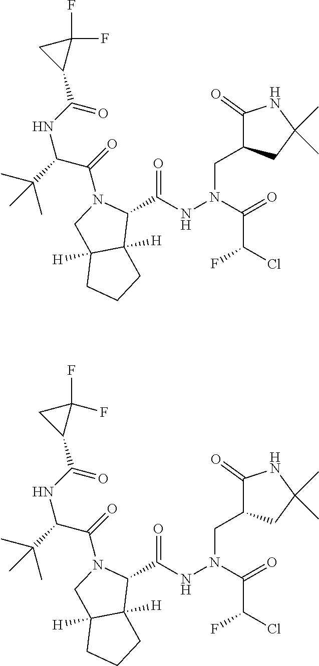

### Step 1: Preparation of benzyl (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylate

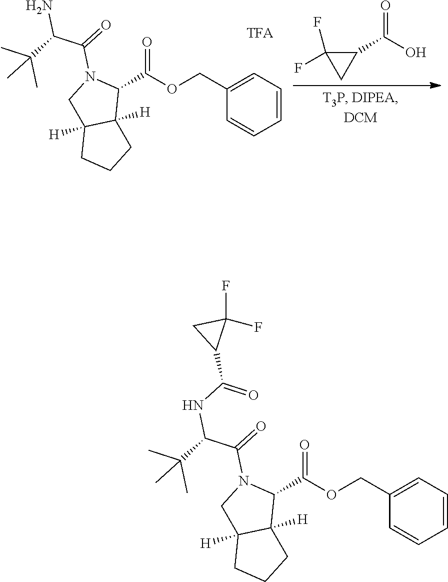

To a solution of (1S)-2,2-difluorocyclopropanecarboxylic acid (274 mg, 2.24 mmol, Pharmablock, CAS: 1883301-82-3) and benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate;2,2,2-trifluoroacetic acid (1.06 g, 2.24 mmol, Intermediate 4) in DCM (15 mL) was added DIPEA (1450 mg, 11.22 mmol). After cooling to 0° C., to the mixture was added T₃P (1570 mg, 2.47 mmol, 50% purity in EtOAc) dropwise. The mixture was stirred at 25° C. for 1 h. The resulting mixture was concentrated in vacuum to and the residue was purified by silica gel column eluted with PE/EtOAc=3/1 to afford benzyl (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylate (900 mg) as yellow oil.

MS obsd. (ESI⁺) [(M+H)⁺]: 463.2.

### Step 2: Preparation of (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylic acid

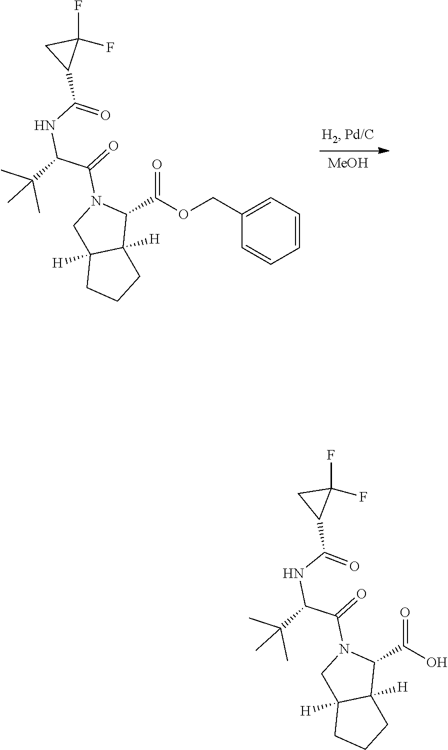

To a solution of benzyl (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylate (900 mg, 1.95 mmol) in THF (20 mL) was added 90 mg of 10% Pd on activated charcoal. The mixture was degassed under vacuum and purged with hydrogen for three times. The resulting mixture was stirred at 25° C. under a hydrogen balloon for 1 h. The mixture was filtered and the filtrate was concentrated in vacuum to afford (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylic acid (600 mg) as a yellow foam.

### Step 3: Preparation of tert-butyl N-[[(3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carbonyl]amino]-N-[rac-(5,5-dimethyl-2-oxo-pyrrolidin-3-yl)methyl]carbamate

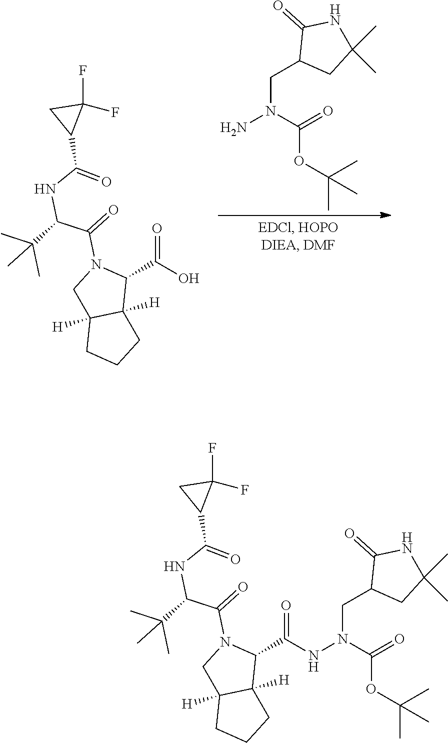

To a solution of (3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carboxylic acid (600 mg, 1.61 mmol) in DMF (10 mL) was added DIPEA (521 mg, 4.03 mmol), EDCI (402 mg, 2.09 mmol), HOPO (233 mg, 2.09 mmol) and tert-butyl N-amino-N-[(5,5-dimethyl-2-oxo-pyrrolidin-3-yl)methyl]carbamate (415 mg, 1.61 mmol) at 0° C. The mixture was stirred at 25° C. for 12 h. The reaction mixture was diluted with EtOAc (200 mL), washed with 1 N HCl (50 mL), brine (60 mL×2), dried over Na₂SO₄ and concentrated in vacuum to afford tert-butyl N-[[(3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carbonyl]amino]-N-[rac-(5,5-dimethyl-2-oxo-pyrrolidin-3-yl)methyl]carbamate (910 mg) as a yellow foam.

MS obsd. (ESI⁺) [(M+H)⁺]: 612.4.

### Step 4: Preparation of N-[(1S)-1-[(3S,3aS,6aR)-3-[[rac-(5,5-dimethyl-2-oxo-pyrrolidin-3-yl)methylamino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

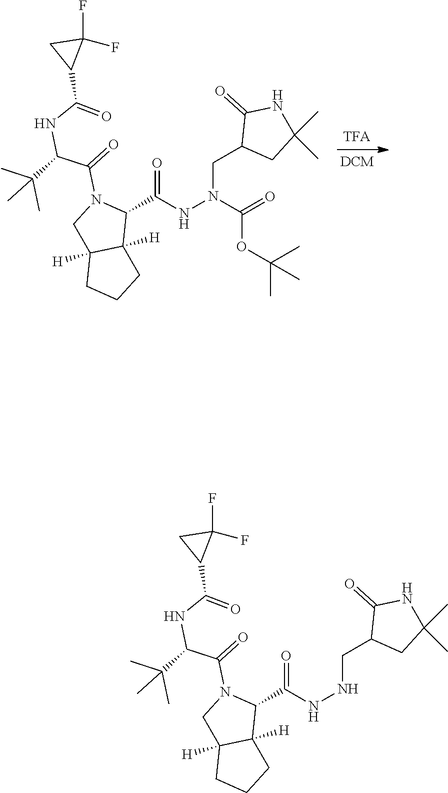

A solution of tert-butyl N-[[(3S,3aS,6aR)-2-[(2S)-2-[(2,2-difluorocyclopropanecarbonyl)amino]-3,3-dimethyl-butanoyl]-3,3a,4,5,6,6a-hexahydro-1H-cyclopenta[c]pyrrole-3-carbonyl]amino]-N-[rac-(5,5-dimethyl-2-oxo-pyrrolidin-3-yl)methyl]carbamate (900 mg, 1.47 mmol) in DCM (10 mL) and TFA (3 mL) was stirred at 25° C. for 1 h. The reaction mixture was concentrated in vacuum to give a residue. The residue was purified by reverse flash chromatography (120 g Flash Column; Welch Ultimate XB_C18 20-40 μm; 35 min; 75 mL/min, ACN-Water, 0.1% HCl) to afford N-[(1S)-1-[(3S,3aS,6aR)-3-[[rac-(5,5-dimethyl-2-oxo-pyrrolidin-3-yl)methylamino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (510 mg) as a white solid.

MS obsd. (ESI⁺) [(M+H)⁺]: 512.7.

### Step 5: Preparation of (1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide and (1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

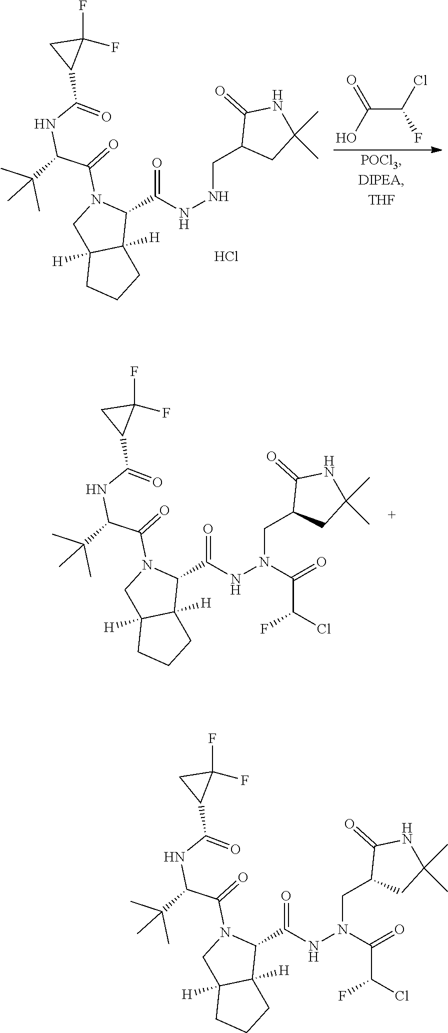

To a solution of (2S)-2-chloro-2-fluoro-acetic acid (260 mg, 1.51 mmol, Intermediate 2) in DCM (15 mL) was added POCl₃ (210 mg, 1.37 mmol). The mixture was stirred at 25° C. for 1 h. Then the resulting solution was added dropwise to a solution of N-[(1S)-1-[(3S,3aS,6aR)-3-[[rac-(5,5-dimethyl-2-oxo-pyrrolidin-3-yl)methylamino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (350 mg, 0.68 mmol) and DIPEA (884 mg, 6.84 mmol) in DCM (15 mL) at −5° C. Then the mixture was stirred at the same temperature for 30 min. The reaction was quenched with MeOH (10 mL) and acidified by 4 N HCl/dioxane to pH=5 at −10° C. Then the resulting mixture was concentrated in vacuum at 30° C. and the residue was purified by reversed flash chromatography (condition: 120 g Flash Column Welch Ultimate XB_C18 20-40 μm; 120 A, water (0.1% FA)-ACN, 0˜60%, 60 mL/min) to afford a mixture of (1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide and (1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide. The mixture was split by prep-SFC (Method Column: DAICEL CHIRALPAK IC (250 mm*30 mm, 10 □m); Condition Neu-MeOH, Begin B 25, End B 25, Gradient Time (min) 3.3 100% B Hold Time (min) Flow Rate (mL/min): 150) to afford Example 2a (retention time=1.93 min) and Example 2b (retention time=3.67 min) as white solids.

### Example 2a

¹H NMR (400 MHz, DMSO-d₆) δ: ppm 11.05-10.69 (m, 1H), 8.45 (d, J=8.4 Hz, 1H), 7.92-7.85 (m, 1H), 7.20-6.38 (m, 1H), 4.85 (d, J=8.8 Hz, 1H), 4.01-3.94 (m, 1H), 3.90-3.67 (m, 3H), 3.55-3.45 (m, 1H), 2.92-2.84 (m, 1H), 2.78-2.69 (m, 2H), 2.62-2.56 (m, 1H), 2.11-1.98 (m, 1H), 1.92-1.78 (m, 4H), 1.75-1.55 (m, 4H), 1.42-1.30 (m, 1H), 1.20 (s, 3H), 1.12-1.09 (m, 3H), 0.98-0.96 (m, 9H).

MS obsd. (ESI⁺) [(M+H)⁺]: 606.3.

### Example 2b

¹H NMR (400 MHz, DMSO-d₆) δ: ppm 11.03-10.68 (m, 1H), 8.47-8.36 (m, 1H), 7.92-7.87 (m, 1H), 7.20-6.52 (m, 1H), 4.50-4.43 (m, 1H), 4.10-3.65 (m, 4H), 2.95-2.80 (m, 2H), 2.80-2.70 (m, 2H), 2.64-2.59 (m, 1H), 2.15-2.07 (m, 1H), 1.90-1.77 (m, 4H), 1.75-1.50 (m, 4H), 1.40-1.33 (m, 1H), 1.21 (s, 3H), 1.45 (d, J=12.0 Hz, 3H), 0.97 (s, 9H).

MS obsd. (ESI⁺) [(M+H)⁺]: 606.3.

### Example 3

### (1S)—N-[(1S)-1-[(3S,3aS,6aR)-3-[[[(2R)-2-chloro-2-fluoro-acetyl]-[rac-(5,5-dimethyl-2-oxo-pyrrolidin-3-yl)methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

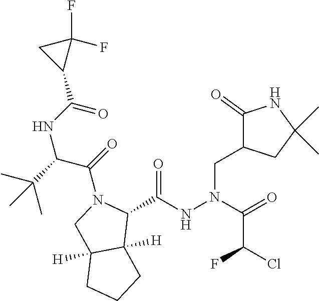 [SMILES: CC1(C)CC(CN(NC(=O)[C@@H]2[C@H]3CCC[C@H]3CN2C(=O)[C@@H](NC(=O)[C@@H]2CC2(F)F)C(C)(C)C)C(=O)[C@H](F)Cl)C(=O)N1]

The title compound was prepared in analogy to the procedure described for the preparation of Example 2a and Example 2b, by using (2R)-2-chloro-2-fluoro-acetic acid (Intermediate 1) instead of (2S′)-2-chloro-2-fluoro-acetic acid (Intermediate 2), to afford the racemic product (1S)—N-[(1S)-1-[(3S,3aS,6aR)-3-[[[(2R)-2-chloro-2-fluoro-acetyl]-[rac-(5,5-dimethyl-2-oxo-pyrrolidin-3-yl)methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide as a white solid.

¹H NMR (400 MHz, DMSO-d₆) δ: ppm 11.10-11.04 (m, 1H), 8.49-8.35 (m, 1H), 7.98-7.82 (m, 1H), 7.00 (d, J=50.4 Hz, 1H), 4.51-4.44 (s, 1H), 4.00-3.84 (m, 2H), 3.79-3.65 (m, 2H), 3.46-3.41 (m, 1H), 2.95-2.86 (m, 1H), 2.78-2.70 (m, 1H), 2.61-2.56 (m, 2H), 2.17-2.00 (m, 1H), 1.93-1.72 (m, 6H), 1.68-1.54 (m, 2H), 1.40-1.31 (m, 1H), 1.21 (s, 3H), 1.15 (d, J=7.2 Hz, 3H), 0.96 (s, 9H).

MS obsd. (ESI⁺) [(M+H)⁺]: 606.2.

The following examples can be made in analogy to the examples described hereinbefore:

| Example | Structure | Name | NMR | Mass |
|---|---|---|---|---|
| 4 | 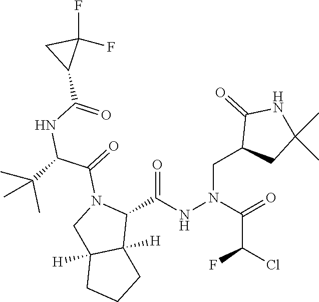 [SMILES: CC1(C)C[C@@H](CN(NC(=O)[C@@H]2[C@H]3CCC[C@H]3CN2C(=O)[C@@H](NC(=O)[C@@H]2CC2(F)F)C(C)(C)C)C(=O)[C@H](F)Cl)C(=O)N1] | (1S)-N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-d6) δ ppm11.09 (s, 1 H), 8.47 (d, J = 8.4 Hz, 1 H), 7.91 (s, 1 H), 7.00 (d, J = 50.8 Hz, 1 H), 4..48 (d, J = 8.4 Hz, 1 H), 3.94 (d, J = 6.4 Hz, 1 H), 3.88 (dd, J = 10.4 Hz, 7.2 Hz, 1 H), 3.74 (dd, J = 14.0 Hz, 10.4 Hz, 1 H), 3.68 (dd, J = 10.4 Hz, 2.8 Hz, 1 H), 3.41 (dd, J = 13.6 Hz, 3.6 Hz, 1 H), 2.94-2.85(m, 1 H), 2.77- 2.67 (m, 2 H), 2.57-2.52 (m, 2 H), 2.02 (dd, J = 12.4 Hz, 8.4 Hz, 1 H), 1.88-1.74 (m, 5 H), 1.67- 1.53 (m, 2 H), 1.40-1.31 | MS obsd. (ESI⁺) [(M + H)⁺]: 606.3. |
| | | | (m, 1 H), 1.20 (s, 3 H), | |
| | | | 1.14 ( s, 3 H), 0.95 (s, 9 | |
| | | | H). | |
| 5 | 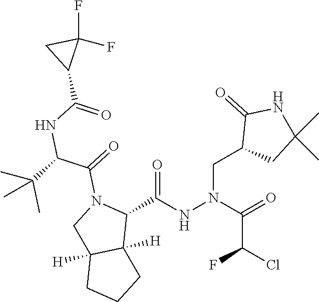 [SMILES: CC1(C)C[C@H](CN(NC(=O)[C@@H]2[C@H]3CCC[C@H]3CN2C(=O)[C@@H](NC(=O)[C@@H]2CC2(F)F)C(C)(C)C)C(=O)[C@H](F)Cl)C(=O)N1] | (1S)-N-[(1S)-1- [(3S,3aS,6aR)-3- [[[(2R)-2-chloro-2- fluoro-acetyl]-[[(3R)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-d6) δ ppm 11.03 (s, 1 H), 8.47 (d, J = 8.4 Hz, 1H), 7.88 (s, 1 H), 7.00 (d, J = 50.4 Hz, 1 H), 4.47 (d, J = 8.4 Hz, 1 H), 3.97-3.93 (m, 1 H), 3.91-3.84 (m, 1 H), 3.74-3.66 (m, 1 H), 3.38-3.33 (m, 1 H), 2.94- 2.85 (m, 1 H), 2.77-2.69 (m, 1 H), 2.61- 2.52 (m, 3 H), 2.13 (dd, J = 12.8 Hz, 8.8 Hz, 1 H), 1.91-1.69 (m, 6 H), 1.67-1.53 (m, 2 H), 1.38-1.28 (m, 1 H), 1.20 (s, 3 H), 1.13 (s, 3 H), 0.95 (s, 9 H). | MS obsd. (ESI⁺) [(M + H)⁺]: 606.3. |
| 6 | 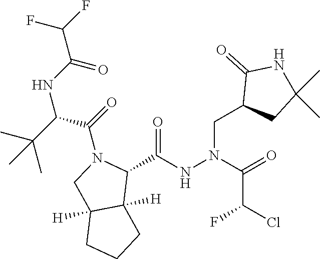 [SMILES: CC1(C)C[C@@H](CN(NC(=O)[C@@H]2[C@H]3CCC[C@H]3CN2C(=O)[C@@H](NC(=O)C(F)F)C(C)(C)C)C(=O)[C@@H](F)Cl)C(=O)N1] | N-[(1S)-1- [(3S,3aS,6aR)-3- [[[(2S)-2-chloro-2- fluoro-acetyl]-[[(3S)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro-acetamide | ¹H NMR (400 MHz, DMSO-d6) δ ppm 10.90 (d, J = 142.4 Hz, 1H), 8.89 (t, J = 8.0 Hz, 1H), 7.89 (d, J = 25.2 Hz, 1H), 7.20-6.15 (m, 2H), 4.50 (d, J = 8.8 Hz, 1H), 4.04-3.96 (m, 1H), 3.91-3.66 (m, 3H), 3.56-3.46 (m, 1H), 2.92- 2.55 (m, 3H), 2.11-1.97 (m, 1H), 1.93-1.78 (m, 2H), 1.78-1.55 (m, 4H), 1.44- 1.32 (m, 1H), 1.20 (s, 3H), 1.11 (d, J = 23.6 Hz, 3H), 0.98 (d, J = 10.4 Hz, 9H). | MS obsd. (ESI⁺) [(M + H)⁺]: 580.3. |
| 7 | 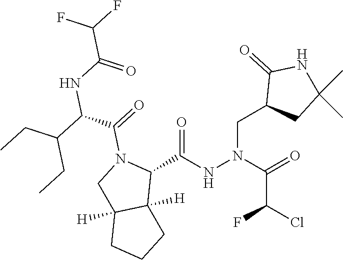 [SMILES: CCC(CC)[C@H](NC(=O)C(F)F)C(=O)N1C[C@@H]2CCC[C@@H]2[C@H]1C(=O)NN(C[C@@H]1CC(C)(C)NC1=O)C(=O)[C@H](F)Cl] | N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2-ethyl- butyl]-2,2-difluoro- acetamide | ¹H NMR (400 MHz, DMSO-d6) δ ppm 11.09 (s, 1H), 9.12 (d, J = 8.4 Hz, 1H), 7.91 (s, 1H), 6.87 (d, J = 50.8 Hz, 1H), 6.26 (t, J = 54.0 Hz, 1H), 4.55 (t, J = 8.4 Hz, 1H), 3.99 (d, J = 6.0 Hz, 1H), 3.88-3.68 (m, 3H), 3.41 (dd, J = 13.6, 4.0 Hz, 1H), 2.85-2.65 (m, 2H), 2.63-2.53 (m, 1H), 2.01 (dd, J = 12.4, 8.4 Hz, 1H), 1.90-1.77 (m, 2H), 1.76-1.67 (m, 3H), 1.66- 1.54 (m, 2H), 1.47-1.35 (m, 2H), 1.32-1.21 (m, 3H), | MS obsd. (ESI⁺) [(M + H)⁺]: 594.3. |
| | | | 1.20 (s, 3H), 1.15 (s, 3H), | |
| | | | 0.82 (t, J = 7.2 Hz, 3H), | |
| | | | 0.76 (t, J = 7.2 Hz, 3H). | |
| 8 | 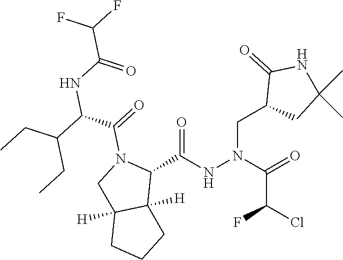 [SMILES: CCC(CC)[C@H](NC(=O)C(F)F)C(=O)N1C[C@@H]2CCC[C@@H]2[C@H]1C(=O)NN(C[C@H]1CC(C)(C)NC1=O)C(=O)[C@H](F)Cl] | N-[(1S)-1- [(3S,3aS,6aR)-3- [[[(2R)-2-chloro-2- fluoro-acetyl]-[[(3R)- 5,5-dimethyl-2-oxo- pyrrolidin-3- yl\|methyl]amino] carbamoyl]- 3,3a,4,5,6,6a- hexahydro-1H- cyclopenta[c]pyrrole- 2-carbonyl]-2-ethyl- butyl]-2,2-difluoro- acetamide | ¹H NMR (400 MHz, DMSO-d6) δ ppm 10.83 (d, J = 117.2 Hz, 1H), 9.08 (dd, J = 36.4 Hz, 8.0 Hz, 1H),7.90 (d, J = 19.6 Hz, 1H) 6.83 (dd, J = 194.0 Hz, 48.8 Hz, 1H), 6.25 (t, J = 53.6 Hz, 1H), 4.70-4.46 (m, 1H), 4.11-4.00 (m, 1H), 3.99-3.92 (m, 1H), 3.98- 3.66 (m, 2H), 3.43-3.34 (m, 1H), 2.94-2.71 (m, 2H), 2.70-2.58 (m, 1H), 2.16- 2.04 (m, 1H), 1.90-1.79 (m, 2H), 1.78-1.52 (m, 5H), 1.50-1.37 (m, 2H), 1.37- | MS obsd. (ESI⁺) [(M + H)⁺]: 594.3. |
| | | | 1.23 (m, 3H), 1.21 (s, 3H), | |
| | | | 1.14 (d, J = 10.4 Hz, 3H), | |
| | | | 0.89-0.81 (m, 3H), 0.80- | |
| | | | 0.72 (m, 3H). | |
| 9 | 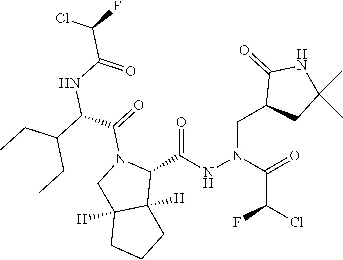 [SMILES: CCC(CC)[C@H](NC(=O)[C@H](F)Cl)C(=O)N1C[C@@H]2CCC[C@@H]2[C@H]1C(=O)NN(C[C@@H]1CC(C)(C)NC1=O)C(=O)[C@H](F)Cl] | (2R)-N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2-ethyl- butyl]-2-chloro-2- fluoro-acetamide | ¹H NMR (400 MHz, DMSO-d6) δ ppm 11.12 (s, 1H), 9.00 (d, J = 8.4 Hz, 1H), 7.94 (s, 1H), 6.82 (dd, J = 50.4 Hz, 46.0 Hz, 2H), 4.54 (t, J = 8.0 Hz, 1H), 4.00 (d, J = 6.0 Hz, 1H), 3.88-3.71 (m, 3H), 3.46- 33.7(m, 1H), 2.84-2.66 (m, 2H), 2.63-2.55 (m, 1H), 2.10-1.98 (m, 1H), 1.92- 1.67 (m, 5H), 1.66-1.51 (m, 2H), 1.49-1.37 (m, 2H), 1.35-1.22 (m, 3H), 1.21 (s, 3H), 1.15 (s, 3H), 0.83 (t, J = 7.2 Hz, 3H), 0.76 (t, J = | MS obsd. (ESI⁺) [(M + H)⁺]: 610.3. |
| | | | 7.2 Hz, 3H). | |
| 10 | 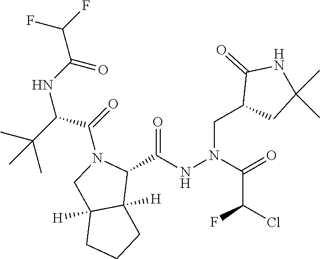 [SMILES: CC1(C)C[C@H](CN(NC(=O)[C@@H]2[C@H]3CCC[C@H]3CN2C(=O)[C@@H](NC(=O)C(F)F)C(C)(C)C)C(=O)[C@H](F)Cl)C(=O)N1] | N-[(1S)-1- [(3S,3aS,6aR)-3- [[[(2R)-2-chloro-2- fluoro-acetyl]-[[(3R)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro-acetamide | ¹H NMR (400 MHz, DMSO-d6) δ ppm 11.07 (s, 1H), 8.96 (d, J = 8.8 Hz, 1H), 7.90 (s, 1H), 6.98 (d, J = 50.4 Hz, 1H), 6.33 (t, J = 54.0 Hz, 1H), 4.50 (d, J = 8.4 Hz, 1H), 4.01-3.93 (m, 1H), 3.93-3.87 (m, 1H), 3.72 (dd, J = 10.4 Hz, 2.4 Hz, 1H), 3.39-3.34 (m, 1H), 3.32-3.31 (m, 1H), 2.79-2.69 (m, 1H), 2.64- 2.54 (m, 2H), 2.13 (dd, J = 12.4 Hz, 8.8 Hz, 1H), 1.92- 1.71 (m, 4H), 1.69-1.55 (m, 2H), 1.42-1.31 (m, 1H), | MS obsd. (ESI⁺) [(M + H)⁺]: 580.3. |
| | | | 1.21 (s, 3H), 1.13 (s, 3H), | |
| | | | 0.96 (s, 9H). | |
| 11 | 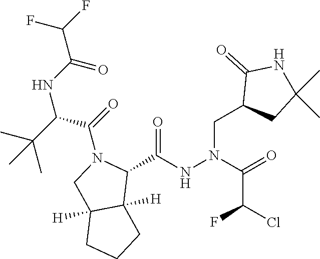 [SMILES: CC1(C)C[C@@H](CN(NC(=O)[C@@H]2[C@H]3CCC[C@H]3CN2C(=O)[C@@H](NC(=O)C(F)F)C(C)(C)C)C(=O)[C@H](F)Cl)C(=O)N1] | N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro-acetamide | ¹H NMR (400 MHz, DMSO-d6) δ ppm 11.14 (s, 1H), 8.96 (d, J =8.8 Hz, 1H), 7.93 (s, 1H), 6.98 (d, J = 50.4 Hz, 1H), 6.34 (d, J = 53.6 Hz, 1H), 4.51 (d, J = 8.4 Hz, 1H), 4.00-3.81 (m, 2H), 3.79-3.65 (m, 2H), 3.46-3.39 (m, 1H), 2.80- 2.65 (m, 2H), 2.61-2.55 (m, 1H), 2.10-1.97 (m, 1H), 1.93-1.84 (m, 1H), 1.83- 1.70 (m, 3H), 1.68-1.52 (m, 2H), 1.42-1.32 (m, 1H), 1.21 (s, 3H), 1.15 (s, 3H), 0.96 (s, 9H). | MS obsd. (ESI⁺) [(M + H)⁺]: 580.3. |
| 12 | 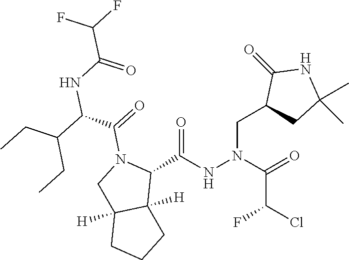 [SMILES: CCC(CC)[C@H](NC(=O)C(F)F)C(=O)N1C[C@@H]2CCC[C@@H]2[C@H]1C(=O)NN(C[C@@H]1CC(C)(C)NC1=O)C(=O)[C@@H](F)Cl] | N-[(1S)-1- [(3S,3aS,6aR)-3- [[[(2S)-2-chloro-2- fluoro-acetyl]-[[(3S)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2-ethyl- butyl]-2,2-difluoro- acetamide | ¹H NMR (400 MHz, DMSO-d6) δ ppm 10.85 (d, J = 147.2 Hz, 1H), 9.16- 9.02 (m, 1H), 7.88 (d, J = 25.6 Hz, 1H), 7.23-6.03 (m, 2H), 4.64-4.50 (m, 1H), 4.06-3.97 (m, 1H), 3.90- 3.68 (m, 3H), 3.56-3.42 (m, 1H), 2.97-2.77 (m, 1H), 2.76-2.57 (m, 2H), 2.10- 1.98 (m, 1H), 1.90-1.79 (m, 2H), 1.78-1.71 (m, 2H), 1.70-1.56 (m, 3H), 1.49- 1.36 (m, 2H), 1.35-1.21 (m, 3H), 1.19 (s, 3H), 1.12 (d, J = 16.4 Hz, 3H), 0.87-0.80 (m, 3H), 0.75 (t, J = 7.2 Hz, 3H). | MS obsd. (ESI⁺) [(M + H)⁺]: 594.4. |
| 13 | 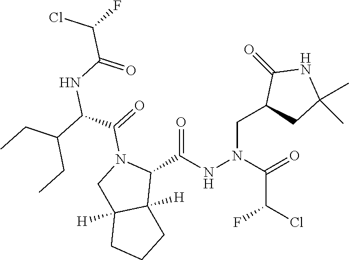 [SMILES: CCC(CC)[C@H](NC(=O)[C@@H](F)Cl)C(=O)N1C[C@@H]2CCC[C@@H]2[C@H]1C(=O)NN(C[C@@H]1CC(C)(C)NC1=O)C(=O)[C@@H](F)Cl] | (2S)-N-[(1S)-1- [(3S,3aS,6aR)-3- [[[(2S)-2-chloro-2- fluoro-acetyl]-[[(3S)- 5,5-dimethyl-2-oxo- pyrrolidin-3- yl]methyl]amino] carbamoyl]-3, 3a,4,5,6,6a- hexahydro-1H- cyclopenta[c]pyrrole- 2-carbonyl]-2-ethyl- butyl]-2-chloro-2- fluoro-acetamide | ¹H NMR (400 MHz, DMSO-d6) δ ppm 11.06- 10.68 (m, 1H), 9.00-8.90 (m, 1H), 7.93-7.87 (m, 1H), 7.14-6.40 (m, 2H), 4.62- 4.51 (m, 1H), 4.06-3.98 (m, 1H), 3.89-3.69 (m, 3H), 3.56-3.44 (m, 1H), 2.97- 2.82 (m, 1H), 2.77-2.58 (m, 2H), 2.11-1.97 (m, 1H), 1.93-1.80 (m, 2H), 1.79- 1.68 (m, 3H), 1.66-1.56 (m, 2H), 1.51-1.37 (m, 2H), 1.36-1.23 (m, 3H), 1.20 (s, 3H), 1.16-1.08 (m, 3H), 0.89-0.81(m, 3H), 0.77 (t, | MS obsd. (ESI⁺) [(M + H)⁺]: 610.3. |
| | | | J = 7.2 Hz, 3H). | |
| 14 | 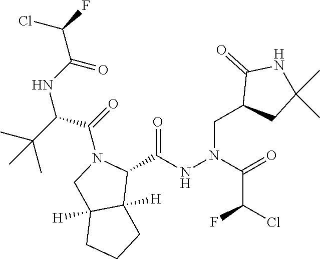 [SMILES: CC1(C)C[C@@H](CN(NC(=O)[C@@H]2[C@H]3CCC[C@H]3CN2C(=O)[C@@H](NC(=O)[C@H](F)Cl)C(C)(C)C)C(=O)[C@H](F)Cl)C(=O)N1] | (2R)-N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]- 3,3a,4,5,6,6a- hexahydro-1H- cyclopenta[c]pyrrole- 2-carbonyl]-2,2- dimethyl-propyl]-2- chloro-2-fluoro- acetamide | ¹H NMR (400 MHz, DMSO-d6) δ ppm 11.12 (s, 1 H), 8.79 (d, J = 9.2 Hz, 1 H), 7.92 (s, 1 H), 7.06- 6.72 (m, 2 H), 4.49 (d, J = 8.8 Hz, 1 H), 3.96 (d, J = 6.4 Hz, 1 H), 3.91 (dd, J = 10.4 Hz, 7.2 Hz, 1 H), 3.75 (dd, J = 13.6 Hz, 10.0 Hz, 1 H), 3.68 (dd, J = 10.4 Hz, 2.8 Hz, 1 H), 3.41 (dd, J = 13.6 Hz, 4 Hz, 1 H), 2.83- 2.67 (m, 2 H), 2.60-2.53 (m, 1 H), 2.02 (dd, J = 12.0 Hz, 8.4 Hz, 1 H), 1.92-1.70 (m, 4 H), 1.68-1.54 (m, 2 | MS obsd. (ESI⁺) [(M + H)⁺]: 596.3. |
| | | | H), 1.42-1.33 (m, 1 H), | |
| | | | 1.20 (s, 3 H), 1.14 (s, 3 H), | |
| | | | 0.95 (s, 9 H). | |
| 15 | 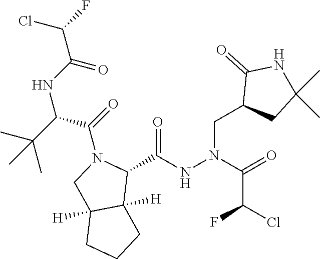 [SMILES: CC1(C)C[C@@H](CN(NC(=O)[C@@H]2[C@H]3CCC[C@H]3CN2C(=O)[C@@H](NC(=O)[C@@H](F)Cl)C(C)(C)C)C(=O)[C@H](F)Cl)C(=O)N1] | (2S)-N-[(1S)-1- [(3S,3aS,6aR)-3- [[[(2R)-2-chloro-2- fluoro-acetyl]-[[(3S)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2- chloro-2-fluoro- acetamide | ¹H NMR (400 MHz, DMSO-d6) δ 11.12 (s, 1H), 8.73 (d, J = 9.1 Hz, 1H), 7.91 (s, 1H), 7.07-6.70 (m, 2H), 4.54-4.39 (m, 1H), 3.97 (d, J = 6.3 Hz, 1H), 3.94-3.87 (m, 1H), 3.78- 3.64 (m, 2H), 3.46-3.41 (m, 1H), 2.80-2.65 (m, 3H), 2.01 (dd, J = 8.6, 12.1 Hz, 1H), 1.92-1.82 (m, 1H), 1.82-1.72 (m, 3H), 1.69- 1.62 (m, 1H), 1.60-1.52 (m, 1H), 1.41-1.31 (m, 1H), 1.20 (s, 3H), 1.14 (s, 3H), 0.96 (s, 9H) | MS obsd. (ESI⁺) [(M + H)⁺]: 596.5. |
| 16 | 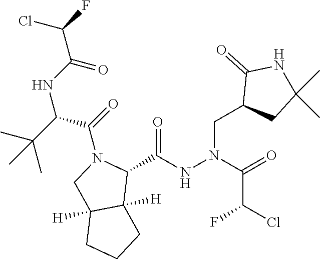 [SMILES: CC1(C)C[C@@H](CN(NC(=O)[C@@H]2[C@H]3CCC[C@H]3CN2C(=O)[C@@H](NC(=O)[C@H](F)Cl)C(C)(C)C)C(=O)[C@@H](F)Cl)C(=O)N1] | (2R)-N-[(1S)-1- [(3S,3aS,6aR)-3- [[[(2S)-2-chloro-2- fluoro-acetyl]-[(3S)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2- chloro-2-fluoro- acetamide | ¹H NMR (400 MHz, DMSO-d6) δ 11.16-10.64 (m, 1H), 8.83-8.70 (m, 1H), 7.96-7.83 (m, 1H), 7.22- 6.35 (m, 2H), 4.55-4.42 (m, 1H), 4.08-3.95 (m, 1H), 3.89-3.81 (m, 1H), 3.93- 3.80 (m, 1H), 2.90-2.81 (m, 1H), 2.79-2.69 (m, 2H), 2.07-2.01 (m, 1H), 1.90- 1.80 (m, 2H), 1.79-1.70 (m, 2H), 1.69-1.58 (m, 2H), 1.44-1.32 (m, 1H), 1.29- 1.22 (m, 2H), 1.21-1.17 (m, 3H), 1.16-1.06 (m, 3H), 1.03-0.90 (m, 9H). | MS obsd. (ESI⁺) [(M + H)⁺]: 596.5. |
| 17 | 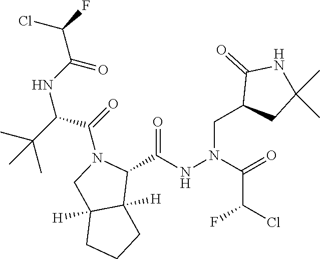 [SMILES: CC1(C)C[C@@H](CN(NC(=O)[C@@H]2[C@H]3CCC[C@H]3CN2C(=O)[C@@H](NC(=O)[C@H](F)Cl)C(C)(C)C)C(=O)[C@@H](F)Cl)C(=O)N1] | (2S)-N-[(1S)-1- [(3S,3aS,6aR)-3- [[[(2S)-2-chloro-2- fluoro-acetyl]-[(3S)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2- chloro-2-fluoro- acetamide | ¹H NMR (400 MHz, DMSO-d6) δ ppm 10.85 (d, J = 146.8 Hz, 1 H), 8.75 - 8.58 (d, J =9.2 Hz, 1 H), 7.88 (d, J = 24.8 Hz, 1 H), 7.20-6.36 (m, 2 H), 4.48 (d, J = 8.8 Hz, 1 H), 4.00 (dd, J = 13.6, 4.8 Hz, 1 H), 3.90- 3.65 (m, 3 H), 3.50 (dd, J = 13.2, 3.6 Hz, 1 H), 2.86- 2.57 (m, 3 H), 2.12-1.97 (m, 1 H), 1.91-1.80 (m, 2 H), 1.77-1.55 (m, 4 H), 1.40-1.32 (m, 1 H), 1.19 (s, 3 H), 1.11 (d, J = 22.4 Hz, 3 H), 0.97 (d, J = 6.4 Hz, 9 H). | MS obsd. (ESI⁺) [(M + H)⁺]: 596.3. |
| 18 | 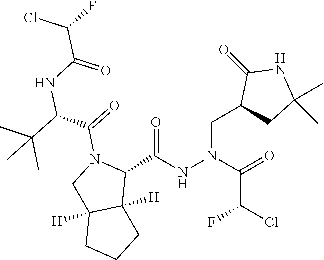 [SMILES: CC1(C)C[C@@H](CN(NC(=O)[C@@H]2[C@H]3CCC[C@H]3CN2C(=O)[C@@H](NC(=O)[C@@H](F)Cl)C(C)(C)C)C(=O)[C@@H](F)Cl)C(=O)N1] | N-[(1S)-1- [(3S,3aS,6aR)-3- [[[(2S)-2-chloro-2- fluoro-acetyl]-[[(3R)- 5,5-dimethyl-2-oxo- pyrrolidin-3- yl]methyl]amino] carbamoyl]-3,3a,4,5,6,6a- hexahydro-1H- cyclopenta[c]pyrrole- 2-carbonyl]-2-ethyl- butyl]-2,2-difluoro- acetamide | ¹H NMR (400 MHz, DMSO-d6) δ ppm 11.03 (s, 1H), 9.14 (d, J = 8.4 Hz, 1H), 7.89 (s, 1H), 6.87 (d, J = 50.8 Hz, 1H), 6.26 (t, J = 54.0 Hz, 1H), 4.54 (t, J = 8.4 Hz, 1H), 4.01 (d, J = 6.0 Hz, 1H), 3.92 (dd, J =14.0, 3.6 Hz, 1H), 3.86-3.73 (m, 2H), 3.39-3.34 (m, 1H), 2.82-2.71 (m, 1H), 2.65- 2.53 (m, 2H), 2.12 (dd, J = 12.4, 8.4 Hz, 1H), 1.91- 1.67 (m, 5H), 1.66-1.53 (m, 2H), 1.47-1.34 (m, 2H), 1.34-1.22 (m, 3H), 1.20 (s, | MS obsd. (ESI⁺) [(M + H)⁺]: 594.3. |
| | | | 3H), 1.13 (s, 3H), 0.82 (t, | |
| | | | J = 7.2 Hz, 3H), 0.82 (t, J = | |
| | | | 7.6 Hz, 3H). | |
| 19 | 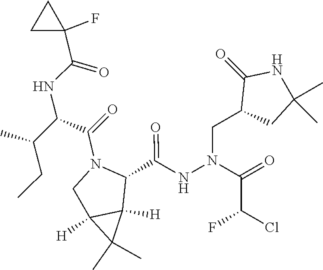 [SMILES: CC[C@H](C)[C@H](NC(=O)C1(F)CC1)C(=O)N1C[C@H]2[C@@H]([C@H]1C(=O)NN(C[C@H]1CC(C)(C)NC1=O)C(=O)[C@@H](F)Cl)C2(C)C] | N-[(1S,2S)-1- [(1R,2S,5S)-2-[[[(2S)- 2-chloro-2-fluoro- acetyl]-[[(3R)-5,5- dimethyl-2-oxo- pyrrolidin-3- yl]methyl]amino] carbamoyl]-6,6-dimethyl- 3-azabicyclo[3.1.0] hexane-3-carbonyl]-2- methyl-butyl]-1- fluoro- cyclopropanecarboxamide | ¹H NMR (400 MHz, DMSO-d6) δ ppm 11.09- 10.79 (m, 1 H), 8.48 (d, J = 8 Hz, 1 H), 7.92-7.80 (m, 1 H), 6.75 (dd, J = 202.4 Hz, 48.8 Hz, 1 H), 4.25-4.12(m, 2 H), 3.95 (d, J = 10.4 Hz, 1 H), 3.87- 3.76 (m, 2 H), 3.47 (dd, J = 14.0 Hz, 3.6 Hz, 1 H), 2.95-2.75 (m, 1 H), 2.12- 1.84 (m, 3H), 1.63-1.57 (m, 2 H), 1.51-1.44 (m, 1 H), 1.31-1.23 (m, 3 H), 1.21-1.14 (m, 5 H), 1.09-1.01 (m, 6 H), 0.89- 0.76 (m, 9H). | MS obsd. (ESI⁺) [(M + H)⁺]: 588.5. |
| 20 | 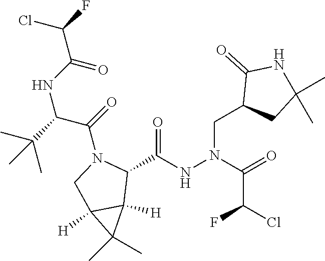 [SMILES: CC1(C)C[C@@H](CN(NC(=O)[C@@H]2[C@@H]3[C@H](CN2C(=O)[C@@H](NC(=O)[C@H](F)Cl)C(C)(C)C)C3(C)C)C(=O)[C@H](F)Cl)C(=O)N1] | (2R)-2-chloro-N- [(1S)-1-[(1R,2S,5S)- 2-[[[(2R)-2-chloro-2- fluoro-acetyl]-[(3S)- 5,5-dimethyl-2-oxo- pyrrolidin-3- yl]methyl]amino] carbamoyl]-6,6-dimethyl- 3-azabicyclo[3.1.0] hexane-3-carbonyl]-2,2- dimethyl-propyl]-2- fluoro-acetamide | ¹H NMR (400 MHz, DMSO-_{d6}) δ ppm 11.20 (s, 1H), 8.76 (d, J = 8.8 Hz, 1H), 7.91 (s, 1H), 6.98- 6.68 (m, 2H), 4.35 (d, J = 8.5 Hz, 1H), 4.14 (s, 1H), 3.95 (dd, J = 10.4 Hz, 5.6 Hz, 1H), 3.84 (br dd, J = 9.6, 13.6 Hz, 1H), 3.76 (br d, J = 10.4 Hz, 1H), 3.40 (br dd, J = 4.2, 13.6 Hz, 2H), 2.80 (dq, J = 4.4, 9.2 Hz, 1H), 2.00 (dd, J = 8.4, 12.0 Hz, 1H), 1.68-1.50 (m, 3H), 1.20 (s, 3H), 1.16 (s, 2H), 1.05 (s, 3H), 0.95 | MS obsd. (ESI⁺) [(M + H)⁺]: 596.2. |
| | | | (s, 9H), 0.90 (s, 3H). | |
| 21 | 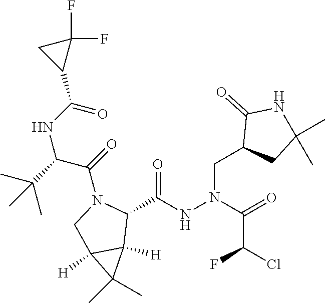 | (1S)-N-[(1S)-1- [(1R,2S,5S)-2- [[[(2R)-2-chloro-2- fluoro-acetyl]-[[(3S)- 5,5-dimethyl-2-oxo- pyrrolidin-3- yl]methyl]amino] carbamoyl]-6,6-dimethyl- 3- azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 11.16 (s, 1 H), 8.44 (d, J = 8.8 Hz, 1 H), 7.90 (s, 1 H), 6.88 (d, J = 50.8 Hz, 1 H), 4.37 (d, J = 8.8 Hz, 1 H), 4.13 (s, 1 H), 3.91 (dd, J = 10.4 Hz, 5.6 Hz, 1 H), 3.85 (dd, J = 14 Hz, 10 Hz, 1 H), 3.76 (d, J = 10.4 Hz, 1H), 3.40 (dd, J = 14.0 Hz, 4.4 Hz, 1 H), 2.91-2.77 (m, 2 H), 2.00 (dd, J = 12.0 Hz, 8.0 Hz, 1 H), 1.88-1.78 (m, 2 H), 1.65-1.55 (m, 2 H), 1.49 (d, J = 7.6 Hz, 1 H), 1.20 (s, 3 H), 1.16 (s, 3 H), 1.04 (s, 3 H), 0.95 (s, 9 H), 0.90 (s, 3 H). | MS obsd. (ESI⁺) [(M + H)⁺]: 606.3. |
| 22 | 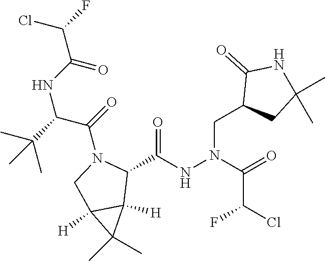 [SMILES: CC1(C)C[C@@H](CN(NC(=O)[C@@H]2[C@@H]3[C@H](CN2C(=O)[C@@H](NC(=O)[C@@H](F)Cl)C(C)(C)C)C3(C)C)C(=O)[C@@H](F)Cl)C(=O)N1] | (2S)-2-chloro-N- [(1S)-1-[(1R,2S,5S)- 2-[[[(2S)-2-chloro-2- fluoro-acetyl]-[[(3S)- 5,5-dimethyl-2-oxo- pyrrolidin-3- yl]methyl]amino] carbamoyl]- 6,6-dimethyl-3- azabicyclo[3.1.0] hexane-3-carbonyl]-2,2- dimethyl-propyl]-2- fluoro-acetamide | ¹H NMR (400 MHz, DMSO-_{d6}) δ ppm 11.22- 10.77 (m, 1H), 8.70 (br dd, J = 9.2, 14.0 Hz, 1H), 7.97-7.80 (m, 1H), 7.15- 6.44 (m, 2H), 4.48-4.35 (m, 1H), 4.26-4.11 (m, 1H), 3.97-3.88 (m, 1H), 3.86-3.67 (m, 2H), 3.58- 3.41 (m, 2H), 2.86-2.73 (m, 1H), 2.10-1.96 (m, 1H), 1.69-1.46 (m, 3H), 1.19 (br s, 3H), 1.15 (s, 1H), 1.08 (s, 1H), 1.05 (br s, 3H), 0.96 (br d, J = 7.6 Hz, 9H), 0.87 (s, 3H). | MS obsd. (ESI⁺) [(M + H)⁺]: 596.2. |
| 23 | 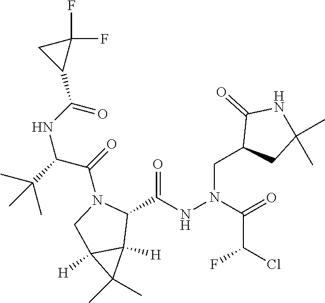 | (1S)-N-[(1S)-1- [(1R,2S,5S)-2-[[[(2S)- 2-chloro-2-fluoro- acetyl]-[[(3S)-5,5- dimethyl-2-oxo- pyrrolidin-3- yl]methyl]amino] carbamoyl]- 6,6-dimethyl-3- azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 10.95 (d, J = 103.6 Hz, 1 H), 8.41 (t, J = 8.0 Hz, 1 H), 7.88 (d, J = 25.2 Hz, 1 H), 6.81 (dd, J = 178.4 Hz, 48.8 Hz, 1 H), 4.38 (d, J = 8.4 Hz, 1 H), 4.16 (d, J = 31.2 Hz, 1 H), 3.91-3.82 (m, 1 H), 3.81- 3.61(m, 2 H), 3.56-3.44 (m, 1H), 2.89-2.75 (m, 2 H), 2.08-1.96 (m, 1 H), 1.87- 1.77 (m, 2H), 1.70-1.43 (m, 3 H), 1.20 (s, 3 H), 1.12 (d, J = 24.8 Hz, 3 H), 1.04 (d, J = 2.0 Hz, 3 H), 0.95 (d, J = 7.2Hz, 9 H), 0.88 (s, 3 H). | MS obsd. (ESI⁺) [(M + H)⁺]: 606.2. |
| 24 | 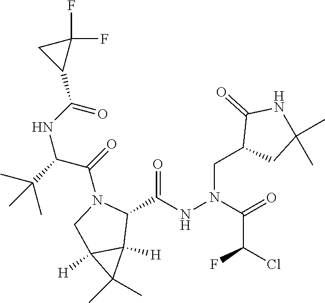 | (1S)-N-[(1S)-1- [(1R,2S,5S)-2- [[[(2R)-2-chloro-2- fluoro-acetyl]-[[(3R)- 5,5-dimethyl-2-oxo- pyrrolidin-3- yl]methyl]amino] carbamoyl]-6,6- dimethyl-3- azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 11.10 (s, 1 H), 8.44 (d, J = 8.4 Hz, 1 H), 7.86 (s, 1 H), 6.87 (d, J = 50.4 Hz, 1 H), 4.36 (t, J = 8.4 Hz, 1 H), 4.15 (s, 1 H), 3.92 (dd, J = 10.4 Hz, 5.6 Hz, 1 H), 3.83 (dd, J = 14.0, 4.0 Hz, 1 H), 3.75 (d, J = 10.4 Hz, 1 H), 3.47 (dd, J = 14.0 Hz, 9.2 Hz, 1 H), 2.91- 2.79 (m, 1 H), 2.74-2.62 (m, 1 H), 2.07 (dd, J = 12.4 Hz, 8.80 Hz, 1H), 1.88- 1.77 (m, 2 H), 1.67-1.57 (m, 2 H), 1.50 (d, J = 7.60 Hz, 1 H), 1.21 (s, 3 H), 1.14 (s, 3 H), 1.04 (s, 3 H), 0.95 (s, 9 H), 0.90 (s, 3 H). | MS obsd. (ESI⁺) [(M + H)⁺]: 606.2. |
| 25 | 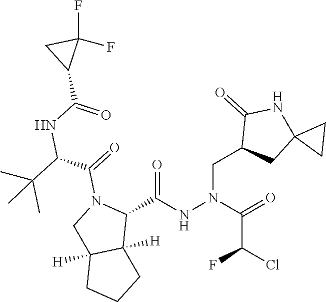 [SMILES: CC(C)(C)[C@H](NC(=O)[C@@H]1CC1(F)F)C(=O)N1C[C@@H]2CCC[C@@H]2[C@H]1C(=O)NN(C[C@@H]1CC2(CC2)NC1=O)C(=O)[C@H](F)Cl] | (1S)-N-[(1S)-1- [(3S,3aS,6aR)-3- [[[(2R)-2-chloro-2- fluoro-acetyl]-[[(6S)- 5-oxo-4- azaspiro[2.4]heptan-6- yl\|methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 11.12 (s, 1 H), 8.48 (d, J = 8.40 Hz, 1 H), 7.89 (s, 1 H), 7.01 (d, J = 50.8 Hz, 1 H), 4.48 (d, J = 8.4 Hz, 1 H), 3.94 (d, J = 6.8 Hz, 1 H), 3.91-3.86 (m, 1 H), 3.73-3.34 (m, 1 H), 2.95-2.85 (m, 1 H), 2.83-2.69 (m, 2 H), 2.58- 2.53 (m, 1H), 2.05 (dd, J = 12.4 Hz, 8.8 Hz, 1 H), 1.94- 1.75 (m, 7H), 1.67-1.60 (m, 1 H), 1.38-1.32 (m, 1 H), 1.24-1.16 (m, 1 H), 0.95 (s, 9 H), 0.78-0.73 (m, 1 H), 0.66-0.56 (m, 3 H). | MS obsd. (ESI⁺) [(M + H)⁺]: 604.3. |
| 26 | 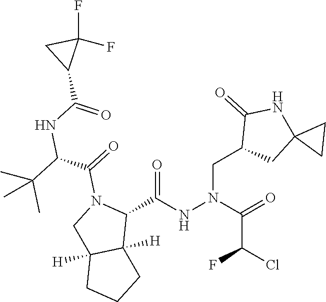 [SMILES: CC(C)(C)[C@H](NC(=O)[C@@H]1CC1(F)F)C(=O)N1C[C@@H]2CCC[C@@H]2[C@H]1C(=O)NN(C[C@H]1CC2(CC2)NC1=O)C(=O)[C@H](F)Cl] | (1S)-N-[(1S)-1- [(3S,3aS,6aR)-3- [[[(2R)-2-chloro-2- fluoro-acetyl]-[[(6R)- 5-oxo-4- azaspiro[2.4]heptan-6- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 11.08 (s, 1 H), 8.48 (d, J = 8.8 Hz, 1 H), 7.84 (s, 1 H), 7.00 (d, J = 50.4 Hz, 1 H), 4.47 (d, J = 8.8 Hz, 1 H), 3.96 (d, J = 6.8 Hz, 1 H), 3.91-3.77 (m, 2 H), 3.69 (dd, J = 10.4, 2.4 Hz, 1 H), 3.52 (dd, J = 13.6 Hz, 9.2 Hz, 1 H), 2.95- 2.82 (m, 1 H), 2.77-2.68 (m, 1 H), 2.64-2.53 (m, 2 H), 2.10 (dd, J = 12.8 Hz, 8.8 Hz, 1 H), 2.00 (dd, J = 12.8 Hz, 8.4 Hz, 1 H), 1.90- 1.71 (m, 6H), 1.66 - 1.58 (m, 1 H), 1.35-1.22 (m, 1 H), 0.95 (s, 9 H), 0.78-0.71 (m, 1 H), 0.61-0.54 (m, 3 H). | MS obsd. (ESI⁺) [(M + H)⁺]: 604.1. |
| 27 | 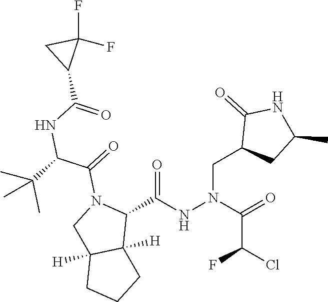 [SMILES: C[C@H]1C[C@@H](CN(NC(=O)[C@@H]2[C@H]3CCC[C@H]3CN2C(=O)[C@@H](NC(=O)[C@@H]2CC2(F)F)C(C)(C)C)C(=O)[C@H](F)Cl)C(=O)N1] | (1S)-N-[(1S)-1- [(3S,3aS,6aR)-3- [[[(2R)-2-chloro-2- fluoro-acetyl]- [[(3S,5S)-5-methyl-2- oxo-pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-a6) δ ppm 11.07 (s, 1H), 8.47 (d, J = 8.8 Hz, 1H), 7.89 (s, 1H), 7.01 (d, J = 50.8 Hz, 1H), 4.49 (d, J = 8.4 Hz, 1H), 3.95 (d, J =6.4 Hz, 1H), 3.91-3.84 (m, 1H), 3.79-3.64 (m, 2H), 3.60-3.49 (m, 1H), 3.48- 3.41 (m, 1H), 2.96-2.83 (m, 1H), 2.77-2.66 (m, 1H), 2.61-2.54 (m, 2H), 2.35- 2.24 (m, 1H), 1.98-1.78 (m, 4H), 1.78-1.73 (m, 2H), 1.70-1.59 (m, 1H), 1.41- 1.21 (m, 2H), 1.12 (d, J = 6.4 Hz, 3H), 0.96 (s, 9H). | MS obsd. (ESI⁺) [(M + H)⁺]: 592.4. |
| 28 | 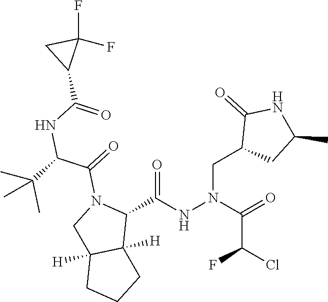 [SMILES: C[C@H]1C[C@H](CN(NC(=O)[C@@H]2[C@H]3CCC[C@H]3CN2C(=O)[C@@H](NC(=O)[C@@H]2CC2(F)F)C(C)(C)C)C(=O)[C@H](F)Cl)C(=O)N1] | (1S)-N-[(1S)-1- [(3S,3aS,6aR)-3- [[[(2R)-2-chloro-2- fluoro-acetyl]- [[(3R,5S)-5-methyl-2- oxo-pyrrolidin-3- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-a6) δ ppm 11.03 (s, 1H), 8.46 (d, J = 8.8 Hz, 1H), 7.83 (s, 1H), 6.99 (d, J = 50.4 Hz, 1H), 4.48 (d, J = 8.4 Hz, 1H), 3.98 (d, J = 6.4 Hz, 1H), 3.91-3.78 (m, 2H), 3.70 (d, J = 10.0 Hz, 1H), 3.65-3.54 (m, 1H), 3.43-3.34 (m, 1H), 2.97- 2.83 (m, 1H), 2.79-2.66 (m, 1H), 2.63-2.54 (m, 1H), 2.04-1.94 (m, 1H), 1.91- 1.75 (m, 7H), 1.75-1.68 (m, 1H), 1.67-1.56 (m, 1H), 1.41-1.27 (m, 1H), 1.07 (d, J = 6.4 Hz, 3H), 0.95 (s, 9H). | MS obsd. (ESI⁺) [(M + H)⁺]: 592.3. |
| 29 | 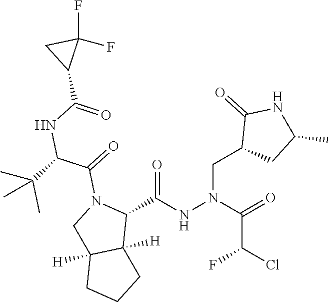 [SMILES: C[C@@H]1C[C@H](CN(NC(=O)[C@@H]2[C@H]3CCC[C@H]3CN2C(=O)[C@@H](NC(=O)[C@@H]2CC2(F)F)C(C)(C)C)C(=O)[C@@H](F)Cl)C(=O)N1] | (1S)-N-[(1S)-1- [(3S,3aS,6aR)-3- [[[(2S)-2-chloro-2- fluoro-acetyl]- [[(3R,5R)-5-methyl- 2-oxo-pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 11.04- 10.67 (m, 1H), 8.48-8.38 (m, 1H), 7.87 (d, J = 7.2 Hz, 1H), 7.25-6.50 (m, 1H), 4.51-4.43 (m, 1H), 4.07-3.95 (m, 1H), 3.93- 3.79 (m, 2H), 3.73-3.66 (m, 1H), 3.56-3.49 (m, 1H), 3.47-3.40 (m, 1H), 2.94- 2.85 (m, 1H), 2.82-2.64 (m, 3H), 2.44-2.39 (m, 1H), 1.89-1.78 (m, 4H), 1.77- 1.60 (m, 3H), 1.41-1.31 (m, 1H), 1.30-1.18 (m, 1H), 1.12 (d, J = 6.0 Hz, 3H), 0.97 (s, 9H). | MS obsd. (ESI⁺) [(M + H)⁺]: 592.1. |
| 30 | 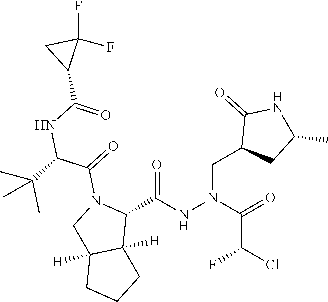 [SMILES: C[C@@H]1C[C@@H](CN(NC(=O)[C@@H]2[C@H]3CCC[C@H]3CN2C(=O)[C@@H](NC(=O)[C@@H]2CC2(F)F)C(C)(C)C)C(=O)[C@@H](F)Cl)C(=O)N1] | (1S)-N-[(1S)-1- [(3S,3aS,6aR)-3- [[[(2S)-2-chloro-2- fluoro-acetyl]- [[(3S,5R)-5-methyl-2- oxo-pyrrolidin-3- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 10.90 (d, J = 139.6 Hz, 1H), 8.46 (d, J = 7.2 Hz, 1H), 7.84 (d, J = 30.8 Hz, 1H), 6.80 (dd, J = 48.8 Hz, 274.0 Hz, 1H), 4.47 (d, J = 8.4 Hz, 1H), 4.01-3.93 (m, 1H), 3.89- 3.73 (m, 2H), 3.72-3.66 (m, 1H), 3.64-3.52 (m, 1H), 3.46-3.37 (m, 1H), 3.02- 2.76 (m, 2H), 2.76-2.62 (m, 2H), 2.08-1.93 (m, 1H), 1.91-1.78 (m, 4H), 1.78- 1.56 (m, 4H), 1.41-1.27 (m, 1H), 1.08 (dd, J = 6.4 Hz, 20.8 Hz, 3H), 1.00-0.91 (m, 9H). | MS obsd. (ESI⁺) [(M + H)⁺]: 592.3. |
| 31 | 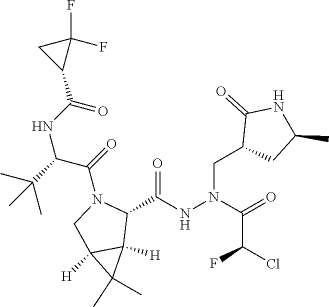 [SMILES: C[C@H]1C[C@H](CN(NC(=O)[C@@H]2[C@@H]3[C@H](CN2C(=O)[C@@H](NC(=O)[C@@H]2CC2(F)F)C(C)(C)C)C3(C)C)C(=O)[C@H](F)Cl)C(=O)N1] | (1S)-N-[(1S)-1- [(1R,2S,5S)-2- [[[(2R)-2-chloro-2- fluoro-acetyl]- [[(3R,5S)-5-methyl-2- oxo-pyrrolidin-3- yl]methyl]amino] carbamoyl]-6,6-dimethyl- 3-azabicyclo[3.1.0] hexane-3-carbonyl]-2,2- dimethyl-propyl]-2,2- difluoro- cyclopropanecarboxamide | ¹H NMR (400 MHz, DMSO-a6) δ ppm 11.11 (s, 1 H), 8.44 (d, J = 8.80 Hz, 1 H), 7.85 (s, 1 H), 6.88 (d, J = 50.0 Hz, 1 H), 4.37 (d, J = 8.4 Hz, 1 H), 4.15 (s, 1 H), 3.91 (dd, J = 10.4 Hz, 5.6 Hz, 1 H), 3.81-3.72 (m, 2 H), 3.66-3.57 (m, 1 H), 3.48 (m, dd, J = 13.6, 8.8 Hz, 1 H), 2.91-2.80 (m, 1 H), 2.63-2.55 (m, 1 H), 2.08-1.98 (m, 1 H), 1.88- 1.72 (m, 3 H), 1.64-1.58 (m, 1 H), 1.51 (d, J = 8.0 Hz, 1 H), 1.08 (d, J = 6.0 Hz, 3 H), 1.04 (s, 3 H), 0.95 (s, 9 H), 0.89 (s, 3H). | MS obsd. (ESI⁺) [(M + H)⁺]: 592.1. |
| 32 | 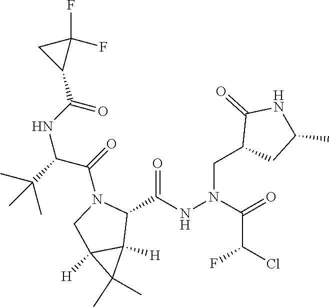 [SMILES: C[C@@H]1C[C@H](CN(NC(=O)[C@@H]2[C@@H]3[C@H](CN2C(=O)[C@@H](NC(=O)[C@@H]2CC2(F)F)C(C)(C)C)C3(C)C)C(=O)[C@@H](F)Cl)C(=O)N1] | (1S)-N-[(1S)-1- [(1R,2S,5S)-2-[[[(2S)- 2-chloro-2-fluoro- acetyl]-[[(3R,5R)-5- methyl-2-oxo- pyrrolidin-3- yl]methyl]amino] carbamoyl]-6,6-dimethyl- 3-azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 10.93 (d, J = 114.8 Hz, 1 H), 8.47- 8.25 (m, 1 H), 7.86 (s, 1 H), 6.91 (dd, J = 99.2 Hz, 48.4 Hz, 1 H), 4.41-4.28 (m, 1 H), 4.17 (d, J = 40.8 Hz, 1 H), 3.91-3.83 (m, 1 H), 3.78-3.74 (m, 1 H), 3.58- 3.47 (m, 2 H), 3.42-3.36 (m, 1 H), 2.88-2.80 (m, 1 H), 2.61-2.55 (m, 1 H), 2.44-2.35 (m, 1 H), 1.87- 1.77 (m, 2 H), 1.70-1.50 (m, 2 H), 1.30-1.19 (m, 1 H), 1.11 (d, J = 6.0 Hz, 3 H), 1.03 (m, 3 H), 0.96 (d, J = 7.2 Hz, 9 H), 0.87 (s, 3 H). | MS obsd. (ESI⁺) [(M + H)⁺]: 592.1. |
| 33 | 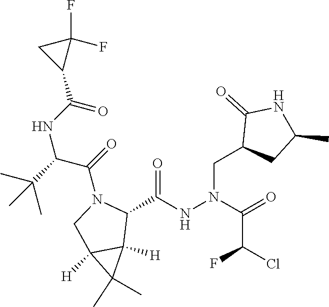 [SMILES: C[C@H]1C[C@@H](CN(NC(=O)[C@@H]2[C@@H]3[C@H](CN2C(=O)[C@@H](NC(=O)[C@@H]2CC2(F)F)C(C)(C)C)C3(C)C)C(=O)[C@H](F)Cl)C(=O)N1] | (1S)-N-[(1S)-1- [(1R,2S,5S)-2- [[[(2R)-2-chloro-2- fluoro-acetyl]- [[(3S,5S)-5-methyl-2- oxo-pyrrolidin-3- yl]methyl]amino] carbamoyl]-6,6-dimethyl- 3-azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 11.14 (s, 1 H), 8.44 (d, J = 8.4 Hz, 1 H), 7.88 (s, 1 H), 6.89 (d, J = 50.4 Hz, 1 H), 4.37 (d, J = 8.8 Hz, 1 H), 4.14 (s, 1 H), 3.92 (dd, J = 10.4 Hz, 5.6 Hz, 1 H), 3.84-3.73 (m, 2 H), 3.58-3.50 (m, 1 H), 3.44 ( dd, J = 13.6, 4.0 Hz, 1 H), 2.90-2.80 (m, 1 H), 2.70-2.60 (m, 1 H), 2.34- 2.24 (m, 1 H), 1.89-1.78 (m, 2 H), 1.66-1.58 (m, 1 H), 1.50 (d, J = 7.6 Hz, 1 H), 1.35-1.25 (m, 1 H), 1.11 (d, J = 6 Hz, 3 H), 1.04 (s, 3 H), 0.95 (s, 9 H), 0.90 (s, 3 H). | MS obsd. (ESI⁺) [(M + H)⁺]: 592.1. |
| 34 | 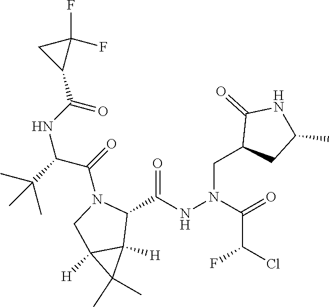 [SMILES: C[C@@H]1C[C@@H](CN(NC(=O)[C@@H]2[C@@H]3[C@H](CN2C(=O)[C@@H](NC(=O)[C@@H]2CC2(F)F)C(C)(C)C)C3(C)C)C(=O)[C@@H](F)Cl)C(=O)N1] | (1S)-N-[(1S)-1- [(1R,2S,5S)-2-[[[(2S)- 2-chloro-2-fluoro- acetyl]-[[(3S,5R)-5- methyl-2-oxo- pyrrolidin-3- yl]methyl]amino] carbamoyl]-6,6- dimethyl-3- azabicyclo[3.1.0] hexane-3-carbonyl]-2,2- dimethyl-propyl]-2,2- difluoro- cyclopropanecarboxamide | ¹H NMR (400 MHz, DMSO-_{d6}) δ ppm 10.96 (d, J = 101.2Hz, 1 H), 8.43 (d, J = 8.4 Hz, 1 H), 7.85 (d, J = 26.8 Hz, 1 H), 6.82 (dd, J = 172.8 Hz, 48.8 Hz, 1 H), 4.38 (d, J = 8 Hz, 1 H), 4.16 (d, J = 35.2 Hz, 1 H), 3.93- 3.83 (m, 1 H), 3.81-3.75 (m, 1 H), 3.70-3.45 (m, 2 H), 3.38-3.25 (m, 1 H), 2.90-2.78 (m, 1 H), 2.77- 2.67 (m, 1 H), 2.07-1.93 (m, 1 H), 1.89-1.76 (m, 2 H), 1.75-1.66 (m, 1 H), 1.65-1.43 (m, 2 H), 1.20- 1.07 (m, 1 H), 1.06-1.00 (m, 5 H),0.95 (s, 9 H), 0.87 (s, 3 H). | MS obsd. (ESI⁺) [(M + H)⁺]: 592.4. |
| 35 | 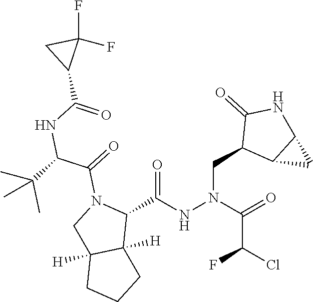 [SMILES: CC(C)(C)[C@H](NC(=O)[C@@H]1CC1(F)F)C(=O)N1C[C@@H]2CCC[C@@H]2[C@H]1C(=O)NN(C[C@H]1C(=O)N[C@@H]2C[C@@H]21)C(=O)[C@H](F)Cl] | (1S)-N-[(1S)-1- [(3S,3aS,6aR)-3- [[[(2R)-2-chloro-2- fluoro-acetyl]- [[(1R,4S,5R)-3-oxo- 2-azabicyclo[3.1.0] hexan-4- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | 1H NMR (400 MHz, DMSO-_{d6}) δ ppm 11.28 (s, 1H), 8.47 (d, J = 8.4 Hz, 1H), 8.33 (s, 1H), 7.07 (d, J = 50.4 Hz, 1H), 4.49 (d, J = 8.4 Hz, 1H), 4.02-3.93 (m, 2H), 3.89 (dd, J = 10.4 Hz, 7.2 Hz, 1H), 3.69 (dd, J = 10.4 Hz, 2.4 Hz, 1H), 3.22 (dd, J = 14.0 Hz, 3.6 Hz, 1H), 3.03-2.96 (m, 1H), 2.95-2.85 (m, 1H), 2.79- 2.68 (m, 1H), 2.56-2.51 (m, 1H), 2.46 (dd, J = 11.2 Hz, 3.2 Hz, 1H), 1.93-1.72 (m, 6H), 1.68-1.58 (m, 1H), 1.38-1.24 (m, 2H), 0.95 (s, 9H), 0.89-0.83 (m, 1H), 0.35-0.27 (m, 1H). | MS obsd. (ESI⁺) [(M + H)⁺]: 590.4. |
| 36 | 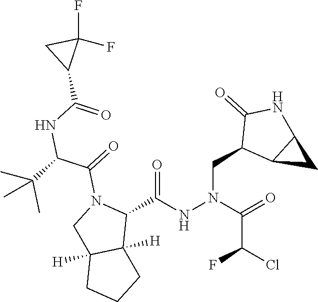 [SMILES: CC(C)(C)[C@H](NC(=O)[C@@H]1CC1(F)F)C(=O)N1C[C@@H]2CCC[C@@H]2[C@H]1C(=O)NN(C[C@H]1C(=O)N[C@H]2C[C@H]21)C(=O)[C@H](F)Cl] | (1S)-N-[(1S)-1- [(3S,3aS,6aR)-3- [[[(2R)-2-chloro-2- fluoro-acetyl]- [[(1S,4S,5S)-3-oxo-2- azabicyclo[3.1.0]hexan-4- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 11.15 (s, 1H), 8.47 (d, J = 8.4 Hz, 1H), 8.29 (s, 1H), 7.05 (d, J = 50.4 Hz, 1H), 4.47 (d, J = 8.4 Hz, 1H), 3.96 (d, J = 6.4 Hz, 1H), 3.87 (dd, J = 10.4 Hz, 7.2 Hz, 1H), 3.71 (dd, J = 10.4 Hz, 2.4 Hz, 1H), 3.61-3.52 (m, 1H), 3.40 (dd, J = 13.6 Hz, 3.2 Hz, 1H), 3.00-2.95 (m, 1H), 2.94-2.85 (m, 2H), 2.76- 2.68 (m, 1H), 2.56-2.51 (m, 1H), 1.92-1.81 (m, 3H), 1.80-1.72 (m, 3H), 1.68- 1.53 (m, 2H), 1.39-1.30 (m, 1H), 0.95 (s, 9H), 0.67-0.60 (m, 1H), 0.32-0.23 (m, 1H). | MS obsd. (ESI⁺) [(M + H)⁺]: 590.1. |
| 37 | 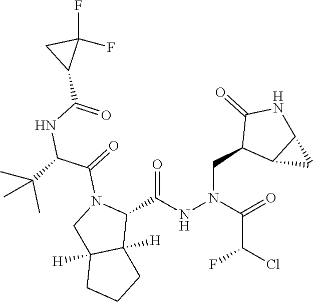 [SMILES: CC(C)(C)[C@H](NC(=O)[C@@H]1CC1(F)F)C(=O)N1C[C@@H]2CCC[C@@H]2[C@H]1C(=O)NN(C[C@H]1C(=O)N[C@@H]2C[C@@H]21)C(=O)[C@@H](F)Cl] | (1S)-N-[(1S)-1- [(3S,3aS,6aR)-3- [[[(2S)-2-chloro-2- fluoro-acetyl]- [[(1R,4S,5R)-3-oxo- 2-azabicyclo[3.1.0] hexan-4- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxa mide | ¹H NMR (400 MHz, DMSO-_{d6}) δ ppm 11.00 (d, J = 169.2 Hz, 1H), 8.44 (d, J = 8.4 Hz, 1H), 8.27 (d, J = 46.8 Hz, 1H), 6.84 (dd, J = 274.8 Hz, 48.4 Hz, 1H), 4.48 (d, J = 8.4 Hz, 1H), 4.06-3.79 (m, 3H), 3.74- 3.67 (m, 1H), 3.01-2.83 (m, 2H), 2.80-2.64 (m, 2H), 2.54-2.39 (m, 1H), 1.94- 1.69 (m, 6H), 1.68-1.46 (m, 2H), 1.42-1.13 (m, 2H), 0.96 (d, J = 3.2 Hz, 9H), 0.88-0.75 (m, 1H), 0.32- 0.02 (m, 1H). | MS obsd. (ESI⁺) [(M + H)⁺]: 590.1. |
| 38 | 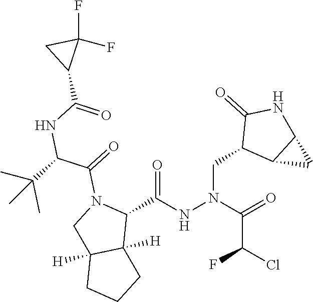 [SMILES: CC(C)(C)[C@H](NC(=O)[C@@H]1CC1(F)F)C(=O)N1C[C@@H]2CCC[C@@H]2[C@H]1C(=O)NN(C[C@@H]1C(=O)N[C@@H]2C[C@H]12)C(=O)[C@H](F)Cl] | (1S)-N-[(1S)-1- [(3S,3aS,6aR)-3- [[[(2R)-2-chloro-2- fluoro-acetyl]- [[(1R,4R,5R)-3-oxo- 2-azabicyclo[3.1.0] hexan-4- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-a6) δppm 11.13 (s, 1H), 8.46 (d, J = 8.8 Hz, 1H), 8.27 (s, 1H), 7.00 (d, J = 50.4 Hz, 1H), 4.48 (d, J = 8.4 Hz, 1H), 3.99 (d, J = 6.4 Hz, 1H), 3.94-3.78 (m, 2H), 3.76-3.68 (m, 1H), 3.27-3.17 (m, 1H), 3.02- 2.96 (m, 1H), 2.95-2.84 (m, 1H), 2.79-2.66 (m, 2H), 2.61-2.53 (m, 1H), 1.93- 1.81 (m, 3H), 1.80-1.67 (m, 4H), 1.65-1.55 (m, 1H), 1.40-1.18 (m, 1H), 0.95 (s, 9H), 0.68-0.59 (m, 1H), 0.29-0.20 (m, 1H). | MS obsd. (ESI⁺) [(M + H)⁺]: 590.2. |
| 39 | 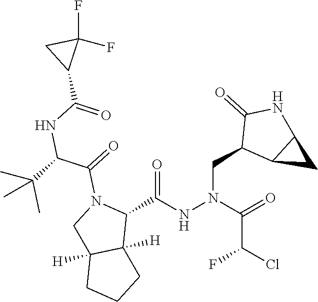 [SMILES: CC(C)(C)[C@H](NC(=O)[C@@H]1CC1(F)F)C(=O)N1C[C@@H]2CCC[C@@H]2[C@H]1C(=O)NN(C[C@H]1C(=O)N[C@H]2C[C@H]21)C(=O)[C@@H](F)Cl] | (1S)-N-[(1S)-1- [(3S,3aS,6aR)-3- [[[(2S)-2-chloro-2- fluoro-acetyl]- [[(1S,4S,5S)-3-oxo-2- azabicyclo[3.1.0] hexan-4- yl]methyl]amino] carbamoyl]- 3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 10.96 (d, J = 154.4 Hz, 1H), 8.46 (t, J = 8.4 Hz, 1H), 8.22 (d, J = 56.4 Hz, 1H), 6.81 (dd, J = 288.8 Hz, 48.4 Hz, 1H), 4.48 (d, J = 8.4 Hz, 1H), 4.07-3.95 (m, 1H), 3.89- 3.80 (m, 1H), 3.75-3.68 (m, 1H), 3.65-3.44 (m, 1H), 3.30-3.24 (m, 1H), 3.00- 2.70 (m, 4H), 2.60-2.51 (m, 1H), 1.92-1.70 (m, 6H), 1.68-1.57 (m, 2H), 1.42- 1.30 (m, 1H), 0.97 (d, J = 8.4 Hz, 9H), 0.70-0.61 (m, 1H), 0.32-0.17 (m, 1H). | MS obsd. (ESI⁺) [(M + H)⁺]: 590.1. |
| 40 | 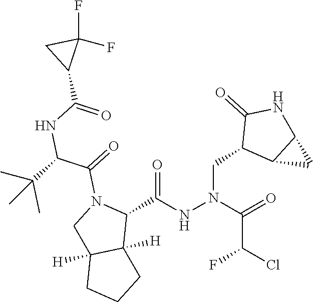 [SMILES: CC(C)(C)[C@H](NC(=O)[C@@H]1CC1(F)F)C(=O)N1C[C@@H]2CCC[C@@H]2[C@H]1C(=O)NN(C[C@@H]1C(=O)N[C@@H]2C[C@H]12)C(=O)[C@@H](F)Cl] | (1S)-N-[(1S)-1- [(3S,3aS,6aR)-3- [[[(2S)-2-chloro-2- fluoro-acetyl]- [[(1R,4R,5R)-3-oxo- 2- azabicyclo[3.1.0] hexan-4- yl]methyl]amino] carbamoyl]-3,3a,4,5,6,6a- hexahydro-1H- cyclopenta[c]pyrrole- 2-carbonyl]-2,2- dimethyl-propyl]-2,2- difluoro- cyclopropanecarboxamide | ¹H NMR (400 MHz, DMSO-_{d6}) δ ppm 10.89 (d, J = 190.0 Hz, 1H), 8.52- 8.37 (m, 1H), 8.31-8.15 (m, 1H), 7.27-6.51 (m, 1H), 4.55-4.39 (m, 1H), 4.14- 3.97 (m, 1H), 3.97-3.77 (m, 2H), 3.76-3.66 (m, 1H), 3.27-3.15 (m, 1H), 3.02- 2.93 (m, 1H), 2.92-2.80 (m, 1H), 2.79-2.65 (m, 2H), 2.64-2.55 (m, 1H), 1.91- 1.80 (m, 3H), 1.80-1.73 (m, 2H), 1.73-1.52 (m, 3H), 1.40-1.15 (m, 1H), 0.95 (d, J = 8.4 Hz, 9H), 0.75-0.57 (m, 1H), 0.32-0.16 (m, 1H). | MS obsd. (ESI⁺) [(M + H)⁺]: 590.3. |

### Biological Example

### Example 41

### SARS-CoV-2 3CL^{pro} Inhibition Assay

The full-length gene encoding SARS-CoV-2 3CL^{pro} was optimized and synthesized for Escherichia coli (E. coli) expression. The method of cloning and producing authentic SARS-CoV-2 3CL^{pro} was followed by the protocol published for SARS-CoV 3CL^{pro} previously (Grum-Tokars V. et al. “Evaluating the 3C-like protease activity of SARS-Coronavirus: Recommendations for standardized assays for drug discovery.” Virus Res. 2008 April; 133(1): 63-73). The protein sequence as follows

SGFRKMAFPSGKVEGCMVQVTCGTTTLNGLWLDDVVYCPRHVICT
SEDMLNPNYEDLLIRKSNHNFLVQAGNVQLRVIGHSMQNCVLKLK
VDTANPKTPKYKFVRIQPGQTFSVLACYNGSPSGVYQCAMRPNFT
IKGSFLNGSCGSVGFNIDYDCVSFCYMHHMELPTGVHAGTDLEGN
FYGPFVDRQTAQAAGTDTTITVNVLAWLYAAVINGDRWFLNRFTT
TLNDFNLVAMKYNYEPLTQDHVDILGPLSAQTGIAVLDMCASLKE
LLQNGMNGRTILGSALLEDEFTPFDVVRQCSGVTFQ

For the SARS-CoV-2 3CL^{pro} assay, 2 μL of 0.02 μM recombinant SARS-CoV-2 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the SARS-CoV 3CL^{pro} was calculated by four parameters equation analysis.

### Example 42

### SARS-CoV 3CL^{pro} Inhibition Assay

The SARS-CoV 3CL^{pro} was expressed in E. coli BL21 (DE3) with the protein sequence of

SGFRKMAFPSGKVEGCMVQVTCGTTTLNGLWLDDTVYCPRHVICT
AEDMLNPNYEDLLIRKSNHSFLVQAGNVQLRVIGHSMQNCLLRLK
VDTSNPKTPKYKFVRIQPGQTFSVLACYNGSPSGVYQCAMRPNHT
IKGSFLNGSCGSVGFNIDYDCVSFCYMHHMELPTGVHAGTDLEGK
FYGPFVDRQTAQAAGTDTTITLNVLAWLYAAVINGDRWFLNRFTT
TLNDFNLVAMKYNYEPLTQDHVDILGPLSAQTGIAVLDMCAALKE
LLQNGMNGRTILGSTILEDEFTPFDVVRQCSGVTFQ.

For the SARS-CoV 3CL^{pro} assay, 2 μL of 0.02 μM recombinant SARS-CoV 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the SARS-CoV 3CL^{pro} was calculated by four parameters equation analysis.

### Example 43

### MERS-CoV 3CL^{pro} Inhibition Assay

The MERS-CoV 3CL^{pro} was expressed in E. coli BL21 (DE3) with the protein sequence of

SGLVKMSHPSGDVEACMVQVTCGSMTLNGLWLDNTVWCPRHVMCP
ADQLSDPNYDALLISMTNHSFSVQKHIGAPANLRVVGHAMQGTLL
KLTVDVANPSTPAYTFTTVKPGAAFSVLACYNGRPTGTFTVVMRP
NYTIKGSFLCGSCGSVGYTKEGSVINFCYMHQMELANGTHTGSAF
DGTMYGAFMDKQVHQVQLTDKYCSVNVVAWLYAAILNGCAWFVKP
NRTSVVSFNEWALANQFTEFVGTQSVDMLAVKTGVAIEQLLYAIQ
QLYTGFQGKQILGSTMLEDEFTPEDVNMQIMGVVMQ

For the MERS-CoV 3CL^{pro} assay, 2 μL of 0.02 μM recombinant MERS-CoV 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the MERS-CoV 3CL^{pro} was calculated by four parameters equation analysis.

### Example 44

### HCoV-229E 3CL^{pro} Inhibition Assay

The HCoV-229E 3CL^{pro} was expressed in E. coli BL21 (DE3) with the protein sequence of

AGLRKMAQPSGFVEKCVVRVCYGNTVLNGLWLGDIVYCPRHVIAS
NTTSAIDYDHEYSIMRLHNFSIISGTAFLGVVGATMHGVTLKIKV
SQTNMHTPRHSFRTLKSGEGFNILACYDGCAQGVFGVNMRTNWTI
RGSFINGACGSPGYNLKNGEVEFVYMHQIELGSGSHVGSSFDGVM
YGGFEDQPNLQVESANQMLTVNVVAFLYAAILNGCTWWLKGEKLF
VEHYNEWAQANGFTAMNGEDAFSILAAKTGVCVERLLHAIQVLNN
GFGGKQILGYSSLNDEFSINEVVKQMFGVNLQ

For the HCoV-229E 3CL^{pro} assay, 2 μL of 0.02 μM recombinant HCoV-229E 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the HCoV-229E 3CL^{pro} was calculated by four parameters equation analysis.

### Example 45

### HCoV-OC43 3CL^{pro} Inhibition Assay

The HCoV-OC43 3CL^{pro} was expressed in E. coli BL21 (DE3) with the protein sequence of

SGIVKMVNPTSKVEPCVVSVTYHNMTLNGLWLDDKVYCPRHVICS
ASDMTNPDYTNLLCVTSSDFTVLFDRLSLTVMSYQMRGCMLVLTV
TLQNSRTPKYTFGVVKPGETFTVLAAYNGKPQGAFHVTMRSSYTI
KGSFLCGSCGSVGYVIMGDCVKFVYMHQLELSTGCHTGTDFNGDF
YGPYKDAQVVQLPIQDYIQSVNFLAWLYAAILNNCNWFIQSDKCS
VEDFNVMALSNGFSQVKSDLVIDALASMTGVSLETLLAAIKRLKN
GFQGRQIMGSCSFEDELTPSDV YQQLAGIKLQ

For the HCoV-OC43 3CL^{pro} assay, 2 μL of 0.02 μM recombinant HCoV-OC43 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the HCoV-OC43 3CL^{pro} was calculated by four parameters equation analysis.

TABLE 1
Activity of Examples and Compounds of present
invention in SARS-CoV-2 3CL^{pro} assay

| Example | IC₅₀ (μM) |
|---|---|
| 1a | 0.048 |
| 1b | 0.013 |
| 2a | 0.029 |
| 2b | 0.918 |
| 3 | 0.023 |
| 4 | 0.011 |
| 5 | 0.081 |
| 6 | 0.030 |
| 7 | 0.007 |
| 8 | 0.129 |
| 9 | 0.007 |
| 10 | 0.031 |
| 11 | 0.009 |
| 12 | 0.009 |
| 13 | 0.009 |
| 14 | 0.009 |
| 15 | 0.011 |
| 16 | 0.022 |
| 17 | 0.016 |
| 18 | 0.012 |
| 19 | 0.019 |
| 20 | 0.024 |
| 21 | 0.014 |
| 22 | 0.031 |
| 23 | 0.054 |
| 24 | 0.087 |
| 25 | 0.012 |
| 26 | 0.012 |
| 27 | 0.006 |
| 28 | 0.030 |
| 29 | 0.036 |
| 30 | 0.008 |
| 31 | 0.025 |
| 32 | 0.065 |
| 33 | 0.006 |
| 34 | 0.008 |
| 35 | 0.010 |
| 36 | 0.013 |
| 37 | 0.017 |
| 38 | 0.027 |
| 39 | 0.029 |
| 40 | 0.064 |

TABLE 2
Activity of Examples and Compounds of present
invention in different coronaviruses

| Example | 229E | MERS | OC43 | SARS |
|---|---|---|---|---|
| 1a | 0.421 | 0.635 | 0.063 | 0.084 |
| 1b | 0.131 | 0.234 | 0.030 | 0.018 |
| 2a | 7.85 | 2.91 | 0.035 | 0.064 |
| 2b | >10 | >10 | 1.16 | 1.72 |
| 3 | 2.52 | 0.850 | 0.037 | 0.028 |
| 4 | 0.838 | 0.518 | 0.021 | 0.015 |
| 5 | 4.101 | 1.241 | 0.052 | 0.137 |
| 6 | >10 | 2.947 | 0.015 | 0.041 |
| 7 | 0.130 | 0.162 | 0.020 | 0.009 |
| 8 | 6.714 | 3.020 | 0.045 | 0.256 |
| 9 | 0.241 | 0.250 | 0.017 | 0.007 |
| 10 | 3.040 | 0.794 | 0.017 | 0.053 |
| 11 | 1.691 | 0.909 | 0.016 | 0.014 |
| 12 | 1.245 | 0.555 | 0.012 | 0.013 |
| 13 | 0.401 | 0.263 | 0.015 | 0.011 |
| 14 | 1.270 | 1.244 | 0.017 | 0.009 |
| 15 | 0.589 | 0.488 | 0.019 | 0.012 |
| 16 | >10.0 | 6.158 | 0.019 | 0.033 |
| 17 | 4.260 | 1.865 | 0.016 | 0.019 |
| 18 | 0.332 | 0.345 | 0.019 | 0.023 |
| 19 | 0.834 | 0.553 | 0.030 | 0.032 |
| 20 | 2.022 | 1.073 | 0.036 | 0.032 |
| 21 | 0.778 | 0.402 | 0.030 | 0.022 |
| 22 | 3.516 | 1.875 | 0.033 | 0.048 |
| 23 | 5.434 | 2.075 | 0.162 | 0.085 |
| 24 | 2.254 | 1.405 | 0.192 | 0.177 |
| 25 | 0.040 | 0.062 | 0.023 | 0.014 |
| 26 | 0.045 | 0.111 | 0.025 | 0.011 |
| 27 | 0.030 | 0.063 | 0.011 | 0.010 |
| 28 | 0.492 | 1.156 | 0.025 | 0.074 |
| 29 | 0.233 | 0.344 | 0.023 | 0.093 |
| 30 | 0.066 | 0.091 | 0.017 | 0.011 |
| 31 | 0.116 | 0.555 | 0.044 | 0.053 |
| 32 | 0.070 | 0.200 | 0.071 | 0.116 |
| 33 | 0.026 | 0.072 | 0.013 | 0.013 |
| 34 | 0.037 | 0.080 | 0.014 | 0.014 |
| 35 | 0.041 | 0.117 | 0.018 | 0.007 |
| 36 | 0.054 | 0.174 | 0.020 | 0.014 |
| 37 | 0.191 | 0.320 | 0.028 | 0.021 |
| 38 | 0.134 | 0.398 | 0.028 | 0.054 |
| 39 | 0.207 | 0.289 | 0.028 | 0.034 |
| 40 | 0.257 | 0.538 | 0.052 | 0.122 |

### Example 46

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 47

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 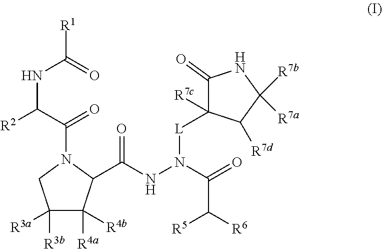
or a pharmaceutically acceptable salt thereof, wherein:
L is C₁-C₆-alkyl;
R¹ is selected from C₃-C₁₀-cycloalkyl, halo-C₃-C₁₀-cycloalkyl, C₆-C₁₄-aryl, 3- to 14-membered heteroaryl, C₁-C₆-alkyl, and halo-C₁-C₆-alkyl;
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C3-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from fluoro and chloro;
R⁶ is selected from hydrogen, chloro, and acyl;
R^{7a} and R^{7b} are both C₁-C₆-alkyl, R^{7c} and R^{7d} are both selected from hydrogen and C₁-C₆-alkyl; or
R^{7a}, R^{7b} and R^{7d} are hydrogen and R^{7c} is C₁-C₆-alkyl; or
R^{7a} is C₁-C₆-alkyl, R^{7b} is hydrogen, and R^{7c} and R^{7d} are both selected from hydrogen and C₁-C₆-alkyl; or
R^{7a} and R^{7b}, taken together with the carbon atom to which they are attached, form a C₃-C₁₀-cycloalkyl and R^{7c} and R^{7d} are both selected from hydrogen and C₁-C₆-alkyl; or
R^{7a} and R^{7d}, taken together with the carbon atoms to which they are attached, form a C₃-C₁₀-cycloalkyl and R^{7b} and R^{7c} are both selected from hydrogen and C₁-C₆-alkyl.

2. The compound of formula (I) according to claim 1, or a pharmaceutically acceptable salt thereof, wherein R¹ is halo-C₃-C₁₀-cycloalkyl or halo-C₁-C₆-alkyl.

3. The compound of formula (I) according to claim 2, or a pharmaceutically acceptable salt thereof, wherein R¹ is selected from 1-fluorocyclopropyl, 2,2-difluorocyclopropyl, CHClF, and CHF₂.

4. The compound of formula (I) according to claim 3, or a pharmaceutically acceptable salt thereof, wherein R¹ is 1-fluorocyclopropyl or 2,2-difluorocyclopropyl.

5. The compound of formula (I) according to claim 1, or a pharmaceutically acceptable salt thereof, wherein R² is C₁-C₆-alkyl.

6. The compound of formula (I) according to claim 5, or a pharmaceutically acceptable salt thereof, wherein R² is selected from tert-butyl, 1-methylpropyl, and 1-ethylpropyl.

7. The compound of formula (I) according to claim 6, or a pharmaceutically acceptable salt thereof, wherein R² is tert-butyl or 1-methylpropyl.

8. The compound of formula (I) according to claim 1, or a pharmaceutically acceptable salt thereof, wherein:
R^{3a} and R^{4a}, taken together with the carbon atoms to which they are attached, form a C3-C₁₀-cycloalkyl; and R^{3b} and R^{4b} are both hydrogen; wherein said C₃-C₁₀-cycloalkyl is optionally substituted with 1 to 2 C₁-C₆-alkyl substituents.

9. The compound of formula (I) according to claim 8, or a pharmaceutically acceptable salt thereof, wherein:
R^{3a} and R^{4a}, taken together with the carbon atoms to which they are attached, form a cyclopropyl or a cyclopentyl; and R^{3b} and R^{4b} are both hydrogen; wherein said cyclopropyl or cyclopentyl is optionally substituted with 1 to 2 methyl substituents.

10. The compound of formula (I) according to claim 9, or a pharmaceutically acceptable salt thereof, wherein the group 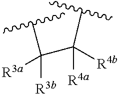
is selected from

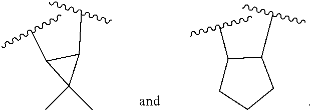

11. The compound of formula (I) according to claim 1, or a pharmaceutically acceptable salt thereof, wherein L is CH₂.

12. The compound of formula (I) according to claim 1, or a pharmaceutically acceptable salt thereof, wherein R⁵ is fluoro and R⁶ is chloro.

13. The compound of formula (I) according to claim 1, or a pharmaceutically acceptable salt thereof, wherein:
R^{7a} and R^{7b} are both C₁-C₆-alkyl and R^{7c} and R^{7d} are both hydrogen; or
R^{7a} and R^{7d}, taken together with the carbon atoms to which they are attached, form a C₃-C₁₀-cycloalkyl and R^{7b} and R^{7c} are both hydrogen; or
R^{7a} is C₁-C₆-alkyl and R^{7b}, R^{7c} and R^{7d} are all hydrogen; or
R^{7a} and R^{7b}, taken together with the carbon atom to which they are attached, form a C₃-C₁₀-cycloalkyl and R^{7c} and R^{7d} are both hydrogen.

14. The compound of formula (I) according to claim 13, or a pharmaceutically acceptable salt thereof, wherein:
R^{7a} and R^{7b} are both methyl and R^{7c} and R^{7d} are both hydrogen; or
R^{7a} and R^{7d}, taken together with the carbon atoms to which they are attached, form a cyclopropyl and R^{7b} and R^{7c} are both hydrogen; or
R^{7a} is methyl and R^{7b}, R^{7c} and R^{7d} are all hydrogen; or
R^{7a} and R^{7b}, taken together with the carbon atom to which they are attached, form a cyclopropyl and R^{7c} and R^{7d} are both hydrogen.

15. The compound of formula (I) according to claim 14, or a pharmaceutically acceptable salt thereof, wherein R^{7a} and R^{7b} are both methyl and R^{7c} is hydrogen.

16. The compound of formula (I) according to claim 1, or a pharmaceutically acceptable salt thereof, selected from:
N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
rac-(1S)-2,2-difluoro-N-[(1S)-2,2-dimethyl-1-[(3S,3aS,6aR)-3-[[(5,5-dimethyl-2-oxo-pyrrolidin-3-yl)methyl-[(2R)-2-chloro-2-fluoro-acetyl]amino]carbamoyl]-3,3a,4,5,6,6a-hexahydro-1H-cyclopenta[c]pyrrole-2-carbonyl]propyl]cyclopropanecarboxamide;
N-[(1S,2S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
(1S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2R)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2-chloro-2-fluoro-acetamide;
N-[(1S)-1-[(3S,3aS,6aR)-3-[[[(2R)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2-chloro-2-fluoro-acetamide;
(2R)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2S)—N-[(1S)-1-[(3S,3aS,6aR)-3-[[[(2R)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2R)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
(2S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-chloro-2-fluoro-acetamide;
N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2R)-2-chloro-N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2-fluoro-acetamide;
(1S)—N-[(1S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(2S)-2-chloro-N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2-fluoro-acetamide;
(1S)—N-[(1S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(6S)-5-oxo-4-azaspiro[2.4]heptan-6-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(6R)-5-oxo-4-azaspiro[2.4]heptan-6-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3S,5S)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(3R,5S)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R,5R)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S,5R)-5-methyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3R,5S)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3R,5R)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2R)-2-chloro-2-fluoro-acetyl]-[[(3 S,5S)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(1R,2S,5S)-2-[[[(2S)-2-chloro-2-fluoro-acetyl]-[[(3S,5R)-5-methyl-2-oxo-pyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1R,4S,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1S,4S,5S)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(1R,4S,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2R)-2-chloro-2-fluoro-acetyl]-[[(1R,4R,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(1S,4S,5S)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide; and
(1S)—N-[(1S)-1-[(3S,3aS,6aR)-3-[[[(2S)-2-chloro-2-fluoro-acetyl]-[[(1R,4R,5R)-3-oxo-2-azabicyclo[3.1.0]hexan-4-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

17. The compound of formula (I) according to claim 16, or a pharmaceutically acceptable salt thereof, selected from:
N-[(1S,2S)-1-[(1R,2S,5S)-2-[[[(2R)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(1S)—N-[(1S)-1-[(3S,3aS,6aR)-3-[[[(2S)-2-chloro-2-fluoro-acetyl]-[[(3S)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3R)-5,5-dimethyl-2-oxo-pyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
rac-(1S)-2,2-difluoro-N-[(1S)-2,2-dimethyl-1-[(3S,3aS,6aR)-3-[[(5,5-dimethyl-2-oxo-pyrrolidin-3-yl)methyl-[(2R)-2-chloro-2-fluoro-acetyl]amino]carbamoyl]-3,3a,4,5,6,6a-hexahydro-1H-cyclopenta[c]pyrrole-2-carbonyl]propyl]cyclopropanecarboxamide;
N-[(1S,2S)-1-[(1R,2S,5S)-2-[[[(2S)-2-chloro-2-fluoro-acetyl]-[[(3S)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide; and
N-[(1S,2S)-1-[(1R,2S,5S)-2-[[[(2S)-2-chloro-2-fluoro-acetyl]-[[(3R)-5,5-dimethyl-2-oxo-pyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

18. A process of manufacturing a compound of formula (I) according to claim 1, or a pharmaceutically acceptable salt thereof, comprising reacting a compound of formula (XI) 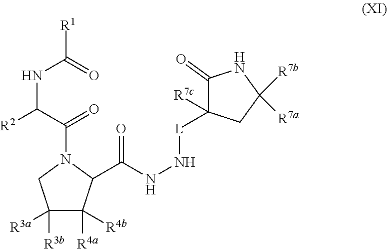
wherein R¹, R², R^{3a}, R^{3b}, R^{4a}, R^{4b}, R^{7a}, R^{7b}, R^{7c}, and L are as defined in claim 1;
with a compound of formula (XI-1), 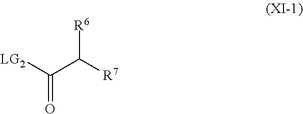
wherein R⁵ and R⁶ are as defined in claim 1 and LG₂ is a leaving group;
in the presence of a coupling reagent and a base, to form said compound of formula (I).

19-20. (canceled)

21. A pharmaceutical composition comprising a compound of formula (I) according to claim 1, or a pharmaceutically acceptable salt thereof, and a therapeutically inert carrier.

22. A method of treatment or prophylaxis of coronavirus infections, said method comprising administering a therapeutically active amount of a compound of formula (I) according to claim 1, or a pharmaceutically acceptable salt thereof, to a subject in need.

23. A method of inhibiting the enzymatic activity of 3C-like proteases, said method comprising contacting a 3C-like protease with a compound of formula (I) according to claim 1, or a pharmaceutically acceptable salt thereof.

24-28. (canceled)

29. The compound of formula (I) according to claim 1, or a pharmaceutically acceptable salt thereof, wherein
L is CH₂;
R⁵ is fluoro; and
R⁶ is chloro.

30. The compound of formula (I) according to claim 29, or a pharmaceutically acceptable salt thereof, wherein
R¹ is selected from 1-fluorocyclopropyl, 2,2-difluorocyclopropyl, CHClF, and CHF₂;
R² is tert-butyl or 1-methylpropyl;
R^{3a} and R^{4a}, taken together with the carbon atoms to which they are attached, form a cyclopentyl or a cyclopropyl substituted with 2 methyl substituents, and R^{3b} and R^{4b} are both hydrogen; and
R^{7a} and R^{7b} are both methyl and R^{7c} and R^{7d} are both hydrogen, or R^{7a} and R^{7d}, taken together with the carbon atoms to which they are attached, form a cyclopropyl and R^{7b} and R^{7c} are both hydrogen.

31. The compound of formula (I) according to claim 29, or a pharmaceutically acceptable salt thereof, wherein
R¹ is 2,2-difluorocyclopropyl;
R² is tert-butyl;
R^{3a} and R^{4a}, taken together with the carbon atoms to which they are attached, form a cyclopentyl or a cyclopropyl substituted with 2 methyl substituents, and R^{3b} and R^{4b} are both hydrogen; and
R^{7a} and R^{7b} are both methyl and R^{7c} and R^{7d} are both hydrogen, or R^{7a} and R^{7d}, taken together with the carbon atoms to which they are attached, form a cyclopropyl and R^{7b} and R^{7c} are both hydrogen.

32. The compound of formula (I) according to claim 1, or a pharmaceutically acceptable salt thereof, wherein
R¹ is selected from 1-fluorocyclopropyl, 2,2-difluorocyclopropyl, CHClF, and CHF₂;
R² is tert-butyl or 1-methylpropyl;
R^{3a} and R^{4a}, taken together with the carbon atoms to which they are attached, form a cyclopentyl or a cyclopropyl substituted with 2 methyl substituents, and R^{3b} and R^{4b} are both hydrogen;
L is CH₂;
R⁵ is fluoro;
R⁶ is chloro; and
R^{7a} and R^{7b} are both methyl and R^{7c} and R^{7d} are both hydrogen, or R^{7a} and R^{7d}, taken together with the carbon atoms to which they are attached, form a cyclopropyl and R^{7b} and R^{7c} are both hydrogen.

33. The compound of formula (I) according to claim 1, or a pharmaceutically acceptable salt thereof, wherein
L is CH₂;
R⁵ is fluoro;
R⁶ is chloro;
R^{7a} and R^{7b} are both methyl; and
R^{7c} and R^{7d} are both hydrogen.

34. The compound of formula (I) according to claim 33, or a pharmaceutically acceptable salt thereof, wherein
R¹ is selected from 1-fluorocyclopropyl, 2,2-difluorocyclopropyl;
R² is tert-butyl or 1-methylpropyl;
R^{3a} and R^{4a}, taken together with the carbon atoms to which they are attached, form a cyclopentyl or a cyclopropyl substituted with 2 methyl substituents; and
R^{3b} and R^{4b} are both hydrogen.


SEQUENCE LISTING (6 sequences)

SEQ ID NO: 1 - protein, 306 aa, SARS-CoV-2 [sequence omitted]

SEQ ID NO: 2 - protein, 306 aa, SARS-CoV
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQ

SEQ ID NO: 3 - protein, 15 aa, synthetic construct
  REGION 1..15: note = Synthetic Construct
KTSAVLQSGFRKMEK

SEQ ID NO: 4 - protein, 306 aa, MERS-CoV
SGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQ

SEQ ID NO: 5 - protein, 302 aa, HCoV-229E
AGLRKMAQPSGFVEKCVVRVCYGNTVLNGLWLGDIVYCPRHVIASNTTSAIDYDHEYSIMRLHNFSIISGTAFLGVVGATMHGVTLKIKVSQTNMHTPRHSFRTLKSGEGFNILACYDGCAQGVFGVNMRTNWTIRGSFINGACGSPGYNLKNGEVEFVYMHQIELGSGSHVGSSFDGVMYGGFEDQPNLQVESANQMLTVNVVAFLYAAILNGCTWWLKGEKLFVEHYNEWAQANGFTAMNGEDAFSILAAKTGVCVERLLHAIQVLNNGFGGKQILGYSSLNDEFSINEVVKQMFGVNLQ

SEQ ID NO: 6 - protein, 302 aa, HCoV-OC43
SGIVKMVNPTSKVEPCVVSVTYHNMTLNGLWLDDKVYCPRHVICSASDMTNPDYTNLLCVTSSDFTVLFDRLSLTVMSYQMRGCMLVLTVTLQNSRTPKYTFGVVKPGETFTVLAAYNGKPQGAFHVTMRSSYTIKGSFLCGSCGSVGYVIMGDCVKFVYMHQLELSTGCHTGTDFNGDFYGPYKDAQVVQLPIQDYIQSVNFLAWLYAAILNNCNWFIQSDKCSVEDFNVMALSNGFSQVKSDLVIDALASMTGVSLETLLAAIKRLKNGFQGRQIMGSCSFEDELTPSDVYQQLAGIKLQ